(19) United States
(12) Patent Application Publication
(10) Pub. No.: US 2025/0098693 A1
(43) Pub. Date: Mar. 27, 2025

(54) METHOD FOR PRODUCING A FERMENTED MILK PRODUCT

(71) Applicant: Kerry Group Services International Ltd, Co. Kerry (IE)

(72) Inventor: Hui Han, Beijing (CN)

(21) Appl. No.: 18/720,137

(22) PCT Filed: Dec. 13, 2022
(86) PCT No.: PCT/EP2022/085602

§ 371 Date: Jun. 14, 2024

(30) Foreign Application Priority Data
Dec. 17, 2021 (WO) PCT/CN2021/139011

(51) Int. Cl. A23C 9/127; A23C 9/123; C12N 9/24

(52) U.S. Cl. CPC A23C 9/1275; A23C 9/1238; C12N 9/2402; C12Y 302/01023; C12Y 302/01108; A23V 2400/123; A23V 2400/249

(57) ABSTRACT

The present invention is in the field of dairy technology. It relates to methods for producing fermented milk products such that the sourness of said product is prevented during its shelf life.

### CROSS-REFERENCE TO RELATED APPLICATIONS

This application is a U.S. National Phase of International Application No. PCT/EP2022/085602, filed on Dec. 13, 2022, and published on Jun. 22, 2023, as WO 2023/110858, which claims the benefit of Chinese Application No. PCT/CN2021/139011, filed on Dec. 17, 2021, the contents of which are hereby incorporated by reference in their entireties for all purposes.

### REFERENCE TO SEQUENCE LISTING

The present application is being filed along with a Sequence Listing in electronic format. The Sequence Listing is provided as a file entitled BRDIT93.008APC_Replacement_Seq_List.XML, which was created and last modified on Dec. 5, 2024, which is 33,898 bytes in size. The information in the electronic Sequence Listing is hereby incorporated by reference in its entirety.

### TECHNICAL FIELD

The present disclosure or invention generally relates to a method to overcome the sourness (or post-acidification) in a fermented milk product, such as yogurt, wherein a lactase having activity over a wide pH range is used, preferably wherein the dosage of lactase is minimized in such a way that lactose is slowly and continuously hydrolyzed over the shelf life of the fermented milk product, thereby counteracting the sourness that may be developed over the shelf life of said product, in particular if the temperature of storage of the product changes over its shelf life. Thus, an increase in sweetness of yoghurt is slowly developed over the shelf life of the fermented milk product, as glucose and galactose are sweeter than lactose.

Surprisingly, the slow and continuously hydrolysis of lactose overcomes the acidic and/or sour taste commonly developed in, for example, yogurt or fresh yogurt. This outcome smoothes the flavor changes of the fermented milk product during its shelf life, which may be specially relevant when cold chain issues arise and the maintaince of yogurt at a constant temperature of 4° C. to 10° C. becomes a challenge.

Alternatively, the method can also be applied to post-pasteurized yogurt supplemented with live bacteria (where the supplementation is made after the pasteurization step), wherein the post-pasteurized yogurt may become more acidic or sour as a result of the added live bacteria.

### BACKGROUND

Fermented milk products, such as yogurts, are well known in the art. These products may be produced from milk or milk base or milk-based substrate that has been standardized with respect to fat and protein content, homogenized and heat treated. Afterwards, the milk (or milk base or milk-based substrate) is inoculated with a starter culture and fermented. Frequently, after fermentation, the product needs to be refrigerated until it is consumed by the consumer.

Unfortunately, the disruption of cold chains used to maintain the fermented milk products refrigerated is common. This situation can lead to public health concerns, for example due to the loss of organoleptic properties (taste, appearance, etc.) of the product and/or the premature spoilage of the product. The disruption of cold chains lead to impaired refrigeration temperatures, which in turn contributes to the production of lactic acid by lactic acid bacteria used to produce the fermented milk product. As a result, the product becomes sour.

The sourness of a fermented milk product is highly undesirable, as the consumer ends up buying yogurts with different tasting profiles depending on the day the product is bought and depending of which point in time the disruption of the cold chain took place.

Therefore, flavor changes of fermented milk products, in particular fresh yoghurt, especially post acidification of the product over the shelf life, are highly problematic for dairy companies, as the damage to the flavor and taste of the product is obvious to the consumer. Thus, there is a need to improve the shelf life flavor performance of fermented milk products, such as yogurt.

Flavor changes of fermented milk products kept in non-refrigerated conditions, i.e. at ambient temperature, may also occur if the product is supplemented with live bacteria after the pasteurization step. The changes in flavor may result from the activity of the added live bacteria.

Several documents disclose fermented milk products and methods thereof where a combination of a starter culture and a beta-galactosidase enzyme is made. However, these documents fail to disclose how changes in the organoleptic properties of fermented milk products, in particular flavor, sourness, sweetness, can be avoided during the shelf life of said product.

WO2015193449 discloses methods for providing a fermented milk product with reduced residual lactose concentration, i.e. a particularly low concentration of lactose. WO2015193459 discloses that cultures fermented in the presence of lactase have a very low residual lactose but a higher residual glucose and galactose concentrations. In particular, Example 5 of WO2015193459 and Examples 2-4 of WO2015193449 disclose the addition of a neutral lactase a method for preparing a yogurt. The neutral lactase used is Ha-Lactase® from Chr. Hansen A/S, which has an optimal pH between 6-8 and is not, therefore, adequate to reduce lactose at a pH below 5. Further, the cited examples show a concentration between less than 0.1 to 5.5 mg/g of lactose on day 1 after fermentation when the neutral lactase is added.

Examples 5-9 of WO2018130630 disclose the production of yogurt using a lactase from Bifidobacterium bifidum. The levels of lactase added to the production of yogurt may be significantly high, approximately 600-3200 LAU/L corresponding to about 210-560 g/ton of milk or milk base or milk-based substrate. The addition of such high concentration of lactase leads to a very low level of lactose in the fermented milk product at the end of fermentation, while the levels of glucose and galactose was very high, all levels compared to the reference samples. As a result, the yogurt produced with lactase disclosed in WO2018130630 has a much higher level of perceived sweetness than the reference samples (yogurt produced without lactase) at the end of fermentation. This observation is transversal to examples 5-9.

### SUMMARY

This disclosure or invention solves the problem of how to avoid flavor changes over the shelf life of a fermented milk product. These changes can occur when the cold chain of the fermented milk product is instable or disrupted in the case of fresh yogurt or when the yogurt or post-pasteurized yogurt is supplemented with live bacteria after the pasteurization step. Thus, the invention relates to the improvement of the overall quality of a fermented milk product, in particular over the shelf life of the product. This is achieved with a low concentration of lactase added to the method for producing a fermented milk product and leading to a product having lactose. Therefore, the fermented milk product (or yogurt) is not a lactose free or a low lactose fermented milk product after the termination of fermentation.

The objects of the disclosure or invention have been achieved when the inventors surprisingly found that adding a low amount or low concentration of lactase leads to a more constant taste during the shelf life of the fermented milk product even if the cold chain is broken or even if live bacteria are added to said product after the pasteurization step. The addition of lactase may be carried out at the start of the fermentation step, during the fermentation step and/or after the fermentation step, preferably at the start of the fermentation step. For example, if the cold chain of a fresh yogurt is broken then it is relevant that the yogurt can keep a good taste at least at early to middle of shelf life (D+7 to D+14). In contrast, high amounts of lactase do not contribute to a constant taste profile of yogurt over time. Instead high amounts of lactase mainly contribute to the hydrolysis of the majority of the lactose available in the milk or milk base or milk-based substrate after 1 day from production which limits the amount of lactose available to be slowly and continuously hydrolysed over the remaining shelf life of the product.

Therefore, it is not a purpose of this disclosure or invention that all or the majority of lactose present in milk (or milk base or milk-based substrate) is hydrolysed immediately after fermentation or at the end of fermentation (D0) or even at day 1 but instead that all or the the majority of lactose remains in the fermented milk product allowing the lactase to hydrolyze lactose over time and at a pH below 5, which is typically the pH of a fermented milk product, such as yogurt.

The present disclosure or invention provides a method for producing a fermented milk product, such as yogurt, preferably fresh yogurt, wherein the taste of the yogurt is kept constant during its shelf life, which may go up to 28 days, by adding low amounts of lactase at the start, during and/or after the fermentation step. Alternatively, this disclosure of invention also provides a method for producing a fermented milk product, such as yogurt, preferably post-pasteurized yogurt also called ambient yogurt, further supplemented with live bacteria after the post-pasteurization step wherein said live bacteria may contribute to changes in the post-acidification of the fermented milk product and, therefore, the sour taste can be overcomed by the addition of a low amount of lactase at the start, during and/or after the fermentation step.

In conclusion, the present disclosure or invention concerns method for producing a fermented milk product comprising the following steps: providing a milk-based substrate comprising lactose; fermenting the milk-based substrate with a lactic acid bacterium until a pH below 5 is reached; and adding a low pH stable beta-galactosidase enzyme or acidic beta-galactosidase enzyme; wherein the beta-galactosidase is present in an initial concentration of less than 200 g beta-galactosidase enzyme/1000 kg of milk-based substrate. Furthermore, the disclosure or invention also concerns a fermented milk product produced by the disclosed method; a composition and the use of a low pH stable beta-galactosidase or acidic beta-galactosidase for maintaining post-acidification, or the sourness and/or sweetness of a fermented milk product constant over the shelf life.

### Definitions

In connection with the present disclosure or invention the following definitions apply.

“Milk” is to be understood as the lacteal secretion obtained by milking of any mammal, such as cows, sheep, goats, buffaloes or camels. In a preferred embodiment, the milk is cow's milk.

The term milk also includes protein/fat solutions made of plant materials, e.g. soy milk, provided lactose is present.

“Milk substrate” or “milk-based substrate” or “milk base” may be any raw and/or processed milk material that can be subjected to fermentation according to the method of the disclosure or invention. Thus, useful milk substrates include, but are not limited to, solutions/-suspensions of any milk or milk like products comprising lactose, preferably comprising at least 0.002% (0.002 g/100 ml) of lactose, such as whole or low-fat milk, skim milk, buttermilk, reconstituted milk powder, condensed milk, dried milk, whey, whey permeate, whey protein concentrate, acid whey, cream, fermented milk products, such as yogurt or cheese. The “milk substrate” or “milk-based substrate” may originate from any mammal, e.g. being substantially pure mammalian milk, or reconstituted milk powder. Typically the term “milk substrate” or “milk-based substrate” refers to a raw or processed milk material that is processed further in order to produce a dairy product. Prior to fermentation, the “milk substrate” may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurization” as used herein means treatment of the milk substrate to reduce or eliminate the presence of live organisms, such as microorganisms, in a milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” as used herein means the conversion of carbohydrates into alcohols or acids through the action of a microorganism. Preferably, fermentation in the methods of the disclosure or invention comprises conversion of lactose to lactic acid. To ferment the milk-based substrate a starter culture is added.

Fermentation processes to be used in production of dairy products are well known and the person of skill in the art will know how to select suitable process conditions, such as temperature, oxygen, amount and characteristics of microorganism(s) and process time. Fermentation conditions are selected so as to support the achievement of the present disclosure or invention, i.e. to obtain a dairy product in solid form (such as a cheese) or liquid form (such as a fermented milk product).

“D” or “D0” as used herein means the day that fermentation is carried out, including the ripening period of the fermented milk product. “D+1”, “D+7”, “D+14”, “D+21” and “D+28” represents days 1, 7, 14, 21 and 28 after fermentation.

“Fermented dairy product” or “fermented milk product” as used herein is to be understood as any dairy product wherein any type of fermentation forms part of the production process. Examples of fermented dairy products are products like yogurt, buttermilk, creme fraiche, quark and fromage frais. A fermented dairy product may be produced by or include steps of any method known in the art. “Starter” or “starter culture” as used in the present context refers to a culture of one or more food-grade microorganisms in particular lactic acid bacteria, which are responsible for the acidification of the milk base. Starter cultures may be fresh, frozen or freeze-dried. It is within the skills of ordinary practitioners to determine the starter culture and amounts to be used.

“Dairy product” as used herein may be any food product wherein one of the major constituents is milk-based. Usually the major constituent is milk-based and in some embodiments, the major constituent is a milk-based substrate which has been treated with an enzyme having beta-galactosidase activity according to a method of the present invention.

A dairy product according to the disclosure or invention may be, e.g., skim milk, low fat milk, whole milk, cream, UHT milk, milk having an extended shelf life, a fermented milk product, cheese, yogurt such as fresh yogurt or post-pasteurized yogurt, butter, dairy spread, butter milk, acidified milk drink, sour cream, whey-based drink, ice cream, condensed milk, dulce de leche or a flavored milk drink.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavoring agents, microbial cultures such as probiotic cultures, salts, sweeteners, sugars, acids, fruit, fruit prep fruit juices, or any other component known in the art as a component of, or additive to, a dairy product.

A post-pasteurized yogurt, also known as ambient yogurt, is heat-treated after completion of the fermentation process to at least inhibit further growth of the bulk of the lactic acid bacteria used in the fermentation process. However, the post-pasteurized yogurt may be further supplemented live bacteria added for health purposes. In this case, it is possible that the bacteria added may interfere with the organoleptic properties of the yogurt, such as the sourness of the yogurt. The post-pasteurized yogurt may be a stirred or drinking yogurt.

“Beta-galactosidase” or “lactase” in the context of the present disclosure or invention is a glycoside hydrolase having the ability to hydrolyze the disaccharide lactose into constituent galactose and glucose monomers. The group of lactases, to which the lactase of the invention belongs, comprises but is not limited to enzymes assigned to subclass EC 3.2.1.108. Enzymes assigned to other subclasses, such as, e.g., EC 3.2.1.23, may also be lactases in the context of the present invention. A lactase in the context of the disclosure or invention may have other activities than the lactose hydrolyzing activity, such as for example a transgalactosylating activity. In the context of the disclosure or invention, the lactose hydrolyzing activity of the lactase may be referred to as its lactase activity or its beta-galactosidase activity. “Beta-galactosidase” and “lactase” can be interchangeably used herein.

“Low pH stable lactase” or “acidic lactase” or “low pH stable beta-galactosidase” or “acidic beta-galactosidase” in the meaning of the present disclosure or invention is a beta-galactosidase or lactase which will be active during the full course of a fermentation process and hence allows conversion of the lactose present in milk to glucose and galactose. Hereby, it is possible to produce a fermented milk product with a reduced content of lactose or a lactose-free product. Also, it is possible to produce a fermented milk product with an increased natural sweetness, as glucose and galactose has a much higher sweetness than lactose. Therefore and contrary to neutral lactases, in particular yeast neutral lactases with are inactivated below pH 5, the low pH stable lactase continues to hydrolyze lactose in fermented milk products down to approximately a pH of 4.5 and also remains at a higher relative activity at both low(er) and high(er) temperatures. Within the meaning of the present disclosure or invention, the terms “low pH stable lactase” or “acidic lactase” or “low pH stable beta-galactosidase” or “acidic beta-galactosidase” can be interchangeably used.

Further, in the context of the present invention, a “low pH stable lactase” or “acidic lactase” or “low pH stable beta-galactosidase” or “acidic beta-galactosidase” may be or is a beta-galactosidase enzyme is selected from a sequence having at least 80% sequence identity to SEQ ID NO: 1, 2, 3, 4, 5, 6, 7, 8, 9, 10, 11, 12, 13, 14, 15, 16, 17, 18, 19, 20 and/or 21; or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21; or least 85% or at least 90% or at least 91% or at least 92% or at least 93% or at least 94% or at least 95% or at least 96% or at least 97% or at least 98% or at least 99% or 100% sequence identity to SEQ ID NO: 21.

“Sequence identity” for amino acids as used herein refers to the sequence identity calculated as (n_{ref}−n_{dif}). 100/n_{ref}, wherein n_{dif} is the total number of non-identical residues in the two sequences when aligned and wherein n_{ref} is the number of residues in one of the sequences. In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

“T” “or t” or “ton” mean tonne. 1 T or 1 t=1000 kg=1 Mg, according to the SI. In the context of the present disclosure, 1 ton means 1 ton of milk or 1 ton of milk base or 1 ton of milk-based substrate.

In the context of the present disclosure “g/ton” means g of beta-galactosidase enzyme/ton of milk or g of beta-galactosidase enzyme/ton of milk base or g of beta-galactosidase enzyme/ton of milk-based substrate.

The use of the terms “a” and “an” and “the” and similar referents in the context of describing the disclosure or invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure or invention and does not pose a limitation on the scope of the disclosure or invention unless otherwise claimed. No language in the specification should be construed as indicating any non-claimed element as essential to the practice of the disclosure or invention.

### BRIEF DESCRIPTION OF THE FIGURES

FIGS. 1 to 4 represent the sensory evaluation obtained when culture 1 is used for producing the fermented milk product and is unsupplemented with lactase (or beta-galactosidase) or supplemented with 30 g/ton, 50 g/ton or 200 g/ton of beta-galactosidase. The fermented milk product was kept at 4° C. Black bars represent sourness and white bars represent sweetness. A, B express statistical significance. The same letter (AA or BB) means there is no significant difference, while a different letter (AB) means significant difference (p<0.05).

FIG. 1 represents the sensory evaluation at D+1;

FIG. 2 represents the sensory evaluation at D+7;

FIG. 3 represents the sensory evaluation at D+14 and

FIG. 4 represents the sensory evaluation at D+21.

FIGS. 5 to 7 represent the sensory evaluation obtained when culture 1 is used for producing the fermented milk product and is unsupplemented with lactase (or beta-galactosidase) or supplemented with 30 g/ton, 50 g/ton or 200 g/ton of beta-galactosidase. The fermented milk product was kept at 25° C. Black bars represent sourness and white bars represent sweetness. A, B express statistical significance. The same letter (AA or BB) means there is no significant difference, while a different letter (AB) means significant difference (p<0.05).

FIG. 5 represents the sensory evaluation at D+7;

FIG. 6 represents the sensory evaluation at D+14;

FIG. 7 represents the sensory evaluation at D+21.

FIGS. 8 to 11 represent the sensory evaluation obtained when culture 2 is used for producing the fermented milk product and is unsupplemented with lactase (or beta-galactosidase) or supplemented with 30 g/ton, 50 g/ton or 200 g/ton of beta-galactosidase. The fermented milk product was kept at 4° C. Black bars represent sourness and white bars represent sweetness. A, B express statistical significance. The same letter (AA or BB) means there is no significant difference, while a different letter (AB) means significant difference (p<0.05).

FIG. 8 represents the sensory evaluation at D+1;

FIG. 9 represents the sensory evaluation at D+7;

FIG. 10 represents the sensory evaluation at D+14 and

FIG. 11 represents the sensory evaluation at D+21.

FIGS. 12 to 14 represent the sensory evaluation obtained when culture 2 is used for producing the fermented milk product and is unsupplemented with beta-galactosidase or supplemented with 30 g/ton, 50 g/ton or 200 g/ton of beta-galactosidase. The fermented milk product was kept at 25° C. Black bars represent sourness and white bars represent sweetness. A, B, C express statistical significance. The same letter (AA or BB) means there is no significant difference, while a different letter (AB) means significant difference (p<0.05).

FIG. 12 represents the sensory evaluation at D+7;

FIG. 13 represents the sensory evaluation at D+14;

FIG. 14 represents the sensory evaluation at D+21.

### DETAILED DESCRIPTION

The present disclosure or invention relates to the recognition that by using a lactase, in particular, a low pH stable lactase (or acidic lactase), in a low concentration in a method for producing a fermented milk product, such as yogurt, wherein low concentration means less than 200 g of lactase/ton of milk or milk base or milk-based substrate, it is possible to obtain a fermented milk product, which simultaneously 1) keeps its organoleptic properties, in particular flavor and/or taste, constant over its shelf life even if the storage temperature is instable, 2) has a reduced lactose content, 3) an increased sweetness and 4) reduced post-acidification, in particular when compared to a fermented milk product prepared under the same conditions but without the addition of said lactase.

### Method for Producing a Fermented Milk Product

This disclosure or invention relates to a method for producing a fermented milk product comprising the following steps:
providing a milk-based substrate comprising lactose,
fermenting the milk-based substrate with a lactic acid bacterium until a pH below 5 is reached;
adding a beta-galactosidase enzyme, preferably adding a low pH stable beta-galactosidase enzyme or acidic beta-galactosidase enzyme;
wherein the beta-galactosidase is present in a concentration, preferably initial concentration, of less than 200 g beta-galactosidase enzyme/1000 kg of milk-based substrate.

Optionally, the beta-galactosidase enzyme may be present in a concentration, preferably an initial concentration, of less than 150 g/1000 kg of milk-based substrate, preferably 5-140 g/1000 kg of milk-based substrate or 5-90 g/1000 kg of milk-based substrate or 10-70 g/1000 kg of milk-based substrate or 30-50 g/1000 kg of milk-based substrate, more preferably 5-35 g/1000 kg of milk-based substrate or 5-30 g/1000 kg of milk-based substrate or 10-30 g/1000 kg of milk-based substrate.

Optionally, the beta-galactosidase enzyme may be selected from a sequence having at least 80% sequence identity to SEQ ID NO: 1, 2, 3, 4, 5, 6, 7, 8, 9, 10, 11, 12, 13, 14, 15, 16, 17, 18, 19, 20 and/or 21, preferably at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21, more preferably least 85% or at least 90% or at least 91% or at least 92% or at least 93% or at least 94% or at least 95% or at least 96% or at least 97% or at least 98% or at least 99% or 100% sequence identity to SEQ ID NO: 21.

Optionally, the lactose concentration of the fermented milk product after the end of fermentation may be more than 0.42 mg_{lactose}/g_{fermented milk product} or more than 0.67 mg_{lactose}/g_{fermented milk product} at the termination of fermentation, preferably more than 1 mg_{lactose}/g_{fermented milk product} or more than 2 mg_{lactose}/g_{fermented milk product}, or more than 3 mg_{lactose}/g_{fermented milk product} or more than 4 mg_{lactose}/g_{fermented milk product} or more than 5.5 mg_{lactose}/g_{fermented milk product}.

Optionally, the milk-based substrate may have at least 1% w_{lactose}/w_{milk-based substrate}, preferably 1-60% w_{lactose}/w_{milk-based substrate}, more preferably 2-50% w_{lactose}/w_{milk-based substrate}, even more preferably 4-40% w_{lactose}/w_{milk-based substrate}.

Optionally, the step of adding a beta-galactosidase enzyme may be carried out before, during and/or after the fermenting step.

Optionally, the lactic acid bacterium is a Streptococcus thermophilus and/or Lactobacillus delbrueckii subsp. bulgaricus.

Optionally, the lactic acid bacterium may be selected from the group consisting of Streptococcus thermophilus strain deposited as DSM22932, DSM22935, DSM24090, DSM24023, DSM32502, DSM32503, DSM32504, DSM32505, DSM32506, DSM32507, DSM25850, DSM25851, DSM26722 and a strain having at least 95% sequence identity to DSM22932, DSM22935, DSM24090, DSM24023, DSM32502, DSM32503, DSM32504, DSM32505, DSM32506, DSM32507, DSM25850, DSM25851, DSM26722.

Streptococcus thermophilus strains deposited as DSM22932, DSM22935, DSM24090, DSM24023 are disclosed in WO2011092300.

Streptococcus thermophilus strains deposited as DSM25850, DSM25851 and DSM26722 are disclosed in WO2013160413.

Streptococcus thermophilus strains deposited as DSM32502, DSM32503, DSM32504, DSM32505, DSM32506 and DSM32507 are disclosed in WO2018220104.

Optionally, the lactic acid bacterium may be selected from the group consisting of Lactobacillus delbrueckii subsp. bulgaricus deposited as DSM24074, DSM26420, DSM26421 and a strain having at least 95% sequence identity to DSM24074, DSM26420, DSM26421.

Lactobacillus delbrueckii subsp. bulgaricus strain deposited as DSM24074 is disclosed in WO2011092300.

Lactobacillus delbrueckii subsp. bulgaricus strain deposited as DSM26420 and DSM26421 are disclosed in WO2013160413.

Optionally, the fermented milk product may be yogurt, fresh yogurt, fruit yogurt, yogurt beverage, yogurt product, stirred yogurt, drinking yogurt, post-pasteurized yogurt, a post-pasteurized stirred yogurt, post-pasteurized drinking yogurt, skyr, Greek yogurt, fortified Greek yogurt, strained Greek yogurt, set yogurt, quark, Dahi, Labneh, or buttermilk.

Optionally, the beta-galactosidase enzyme may have an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

Herein it is also disclosed a method for producing a fermented milk product comprises the following steps:
providing a milk-based substrate comprising lactose,
fermenting the milk-based substrate with a lactic acid bacterium until a pH below 5 is reached;
adding a beta-galactosidase enzyme, preferably adding a low pH stable beta-galactosidase enzyme or acidic beta-galactosidase enzyme;
wherein the beta-galactosidase is present in a concentration, preferably initial concentration, of less than 200 g beta-galactosidase enzyme/1000 kg of milk-based substrate or less than 150 g/1000 kg of milk-based substrate or 5-140 g/1000 kg of milk-based substrate or 5-90 g/1000 kg of milk-based substrate or 10-70 g/1000 kg of milk-based substrate or 30-50 g/1000 kg of milk-based substrate, or 5-35 g/1000 kg of milk-based substrate or 5-30 g/1000 kg of milk-based substrate or 10-30 g/1000 kg of milk-based substrate;
wherein the beta-galactosidase enzyme is selected from a sequence having at least 80% or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21.

Herein is disclosed a method for producing a fermented milk product comprises the following steps:
providing a milk-based substrate comprising lactose,
fermenting the milk-based substrate with a lactic acid bacterium until a pH below 5 is reached;
adding a beta-galactosidase enzyme, preferably adding a low pH stable beta-galactosidase enzyme or acidic beta-galactosidase enzyme;
wherein the beta-galactosidase is present in a concentration, preferably initial concentration, of less than 200 g beta-galactosidase enzyme/1000 kg of milk-based substrate or less than 150 g/1000 kg of milk-based substrate or 5-140 g/1000 kg of milk-based substrate or 5-90 g/1000 kg of milk-based substrate or 10-70 g/1000 kg of milk-based substrate or 30-50 g/1000 kg of milk-based substrate, or 5-35 g/1000 kg of milk-based substrate or 5-30 g/1000 kg of milk-based substrate or 10-30 g/1000 kg of milk-based substrate;
wherein the beta-galactosidase enzyme is selected from a sequence having at least 80% or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21; and
wherein the beta-galactosidase enzyme has an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

Herein is disclosed a method for producing a fermented milk product comprises the following steps:
providing a milk-based substrate comprising lactose,
fermenting the milk-based substrate with a lactic acid bacterium until a pH below 5 is reached;
adding a beta-galactosidase enzyme, preferably adding a low pH stable beta-galactosidase enzyme or acidic beta-galactosidase enzyme;
wherein the beta-galactosidase is present in a concentration, preferably initial concentration, of less than 200 g beta-galactosidase enzyme/1000 kg of milk-based substrate or less than 150 g/1000 kg of milk-based substrate or 5-140 g/1000 kg of milk-based substrate or 5-90 g/1000 kg of milk-based substrate or 10-70 g/1000 kg of milk-based substrate or 30-50 g/1000 kg of milk-based substrate, or 5-35 g/1000 kg of milk-based substrate or 5-30 g/1000 kg of milk-based substrate or 10-30 g/1000 kg of milk-based substrate;
wherein the beta-galactosidase enzyme is selected from a sequence having at least 80% or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21;
wherein the beta-galactosidase enzyme has an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}; and
wherein the fermented milk product is selected from a list consisting of yogurt, fresh yogurt, fruit yogurt, yogurt beverage, yogurt product, stirred yogurt, drinking yogurt, post-pasteurized yogurt, a post-pasteurized stirred yogurt, post-pasteurized drinking yogurt, skyr, Greek yogurt, fortified Greek yogurt, strained Greek yogurt, set yogurt, quark, Dahi, Labneh, buttermilk.

In any of the above embodiments or options within the section “Method for producing a fermented milk product”, the lactose concentration of the fermented milk product after the end of fermentation is or may be more than 0.42 mg_{lactose}/g_{fermented milk product} or more than 0.67 mg_{lactose}/g fermented milk product or more than 1 mg_{lactose}/g_{fermented milk product} or more than 2 mg_{lactose}/g_{fermented milk product}, or more than 3 mg_{lactose}/g_{fermented milk product} or more than 4 mg lactose/g_{fermented milk product} or more than 5.5 mg_{lactose}/g_{fermented milk product}.

In any of the above embodiments or options within the section “Method for producing a fermented milk product”, the milk-based substrate is or may be at least 1% w_{lactose}/w_{milk-based substrate}; or 1-60% w_{lactose}/w_{milk-based substrate}, or 2-50% w_{lactose}/w_{milk-based substrate}, or 4-40% w_{lactose}/w_{milk-based substrate}.

In any of the above embodiments or options within the section “Method for producing a fermented milk product”, the step of adding a beta-galactosidase enzyme is or may be carried out before, during and/or after the fermenting step.

In any of the above embodiments or options within the section “Method for producing a fermented milk product”, the lactic acid bacterium is or may be a Streptococcus thermophilus and/or Lactobacillus delbrueckii subsp. bulgaricus.

In any of the above embodiments or options within the section “Method for producing a fermented milk product”, the lactic acid bacterium is or may be selected from the group consisting of Streptococcus thermophilus strain deposited as DSM22932, DSM22935, DSM24090, DSM24023, DSM32502, DSM32503, DSM32504, DSM32505, DSM32506, DSM32507, DSM25850, DSM25851, DSM26722 and a strain having at least 95% sequence identity to DSM22932, DSM22935, DSM24090, DSM24023, DSM32502, DSM32503, DSM32504, DSM32505, DSM32506, DSM32507, DSM25850, DSM25851, DSM26722. Streptococcus thermophilus strains deposited as DSM22932, DSM22935, DSM24090, DSM24023 are disclosed in WO2011092300. Streptococcus thermophilus strains deposited as DSM25850, DSM25851 and DSM26722 are disclosed in WO2013160413. Streptococcus thermophilus strains deposited as DSM32502, DSM32503, DSM32504, DSM32505, DSM32506 and DSM32507 are disclosed in WO2018220104.

In any of the above embodiments or options within the section “Method for producing a fermented milk product”, the lactic acid bacterium is or may be selected from the group consisting of Lactobacillus delbrueckii subsp. bulgaricus deposited as DSM24074, DSM26420, DSM26421 and a strain having at least 95% sequence identity to DSM24074, DSM26420, DSM26421. Lactobacillus delbrueckii subsp. bulgaricus strain deposited as DSM24074 is disclosed in WO2011092300. Lactobacillus delbrueckii subsp. bulgaricus strain deposited as DSM26420 and DSM26421 are disclosed in WO2013160413.

In any of the above embodiments or options within the section “Method for producing a fermented milk product”, the lactic acid bacterium is or may be selected from the group consisting of: Streptococcus thermophilus strain deposited as DSM22932, DSM22935, DSM24090, DSM24023, DSM32502, DSM32503, DSM32504, DSM32505, DSM32506, DSM32507, DSM25850, DSM25851, DSM26722 and a strain having at least 95% sequence identity to DSM22932, DSM22935, DSM24090, DSM24023, DSM32502, DSM32503, DSM32504, DSM32505, DSM32506, DSM32507, DSM25850, DSM25851, DSM26722; and Lactobacillus delbrueckii subsp. bulgaricus deposited as DSM24074, DSM26420, DSM26421 and a strain having at least 95% sequence identity to DSM24074, DSM26420, DSM26421. Lactobacillus delbrueckii subsp. bulgaricus strain deposited as DSM24074 is disclosed in WO2011092300. Lactobacillus delbrueckii subsp. bulgaricus strain deposited as DSM26420 and DSM26421 are disclosed in WO2013160413.

### Fermented Milk Product Produced by a Method Herein Disclosed

This disclosure or invention also relates to a fermented milk product produced by a method according to any of the above embodiments.

In an embodiment, the fermented milk product produced by any of the methods herein disclosed comprises a beta-galactosidase enzyme, such a low pH stable beta-galactosidase enzyme or acidic beta-galactosidase enzyme, in an concentration of less than 200 g beta-galactosidase enzyme/1000 kg of milk-based substrate or less than 150 g/1000 kg of milk-based substrate, or 5-140 g/1000 kg of milk-based substrate or 5-90 g/1000 kg of milk-based substrate or 10-70 g/1000 kg of milk-based substrate or 30-50 g/1000 kg of milk-based substrate or 5-35 g/1000 kg of milk-based substrate or 5-30 g/1000 kg of milk-based substrate or 10-30 g/1000 kg of milk-based substrate.

In an embodiment, the fermented milk product produced by any of the methods herein disclosed comprises a beta-galactosidase enzyme in an concentration of less than 200 g beta-galactosidase enzyme/1000 kg of milk-based substrate or less than 150 g/1000 kg of milk-based substrate, or 5-140 g/1000 kg of milk-based substrate or 5-90 g/1000 kg of milk-based substrate or 10-70 g/1000 kg of milk-based substrate or 30-50 g/1000 kg of milk-based substrate or 5-35 g/1000 kg of milk-based substrate or 5-30 g/1000 kg of milk-based substrate or 10-30 g/1000 kg of milk-based substrate; wherein the beta-galactosidase enzyme is selected from a sequence having at least 80% or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21.

In an embodiment, the fermented milk product produced by any of the methods herein disclosed comprises a beta-galactosidase enzyme in an concentration of less than 200 g beta-galactosidase enzyme/1000 kg of milk-based substrate or less than 150 g/1000 kg of milk-based substrate, or 5-140 g/1000 kg of milk-based substrate or 5-90 g/1000 kg of milk-based substrate or 10-70 g/1000 kg of milk-based substrate or 30-50 g/1000 kg of milk-based substrate or 5-35 g/1000 kg of milk-based substrate or 5-30 g/1000 kg of milk-based substrate or 10-30 g/1000 kg of milk-based substrate; wherein the beta-galactosidase enzyme is selected from a sequence having at least 80% or at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21; and said beta-galactosidase enzyme has an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

In any of the embodiments disclosed in the present section entitled “Fermented milk product produced by a method herein disclosed”, the fermented milk product is or may be selected from a list consisting of yogurt, fresh yogurt, fruit yogurt, yogurt beverage, yogurt product, stirred yogurt, drinking yogurt, post-pasteurized yogurt, post-pasteurized stirred yogurt, post-pasteurized drinking yogurt, skyr, Greek yogurt, fortified Greek yogurt, strained Greek yogurt, set yogurt, quark, Dahi, Labneh, buttermilk.

### Uses

Further, the disclosure or invention also concerns
the use of a beta-galactosidase, preferably a low pH stable beta-galactosidase or acidic beta-galactosidase, for maintaining sourness and/or sweetness of a fermented milk product constant over shelf life of the fermented milk product as compared to sourness and/or sweetness of a fermented milk product deprived of the beta-galactosidase, preferably wherein the shelf life is less than 30 days or wherein the shelf life is 28 days or less; and/or
the use of a beta-galactosidase, preferably a low pH stable beta-galactosidase or acidic beta-galactosidase, for maintaining post-acidification of a fermented milk product constant over the shelf life of the fermented milk product as compared to post-acidification of a fermented milk product deprived of beta-galactosidase.

Optionally, the use mentioned above concerns a beta-galactosidase enzyme that is or may be selected from a sequence having at least 80% sequence identity to SEQ ID NO: 1, 2, 3, 4, 5, 6, 7, 8, 9, 10, 11, 12, 13, 14, 15, 16, 17, 18, 19, 20 and/or 21, preferably at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21, more preferably least 85% or at least 90% or at least 91% or at least 92% or at least 93% or at least 94% or at least 95% or at least 96% or at least 97% or at least 98% or at least 99% or 100% sequence identity to SEQ ID NO: 21.

Optionally, the use mentioned above concerns a beta-galactosidase enzyme that is or may be added in a concentration, or an initial concentration, of less than 200 g per 1000 kg of milk-based substrate or less than 150 g per 1000 kg of milk-based substrate, preferably 5-140 g per 1000 kg of milk-based substrate or 5-90 g per 1000 kg of milk-based substrate or 10-70 g per 1000 kg of milk-based substrate or 30-50 g per 1000 kg of milk-based substrate, more preferably 5-35 g per 1000 kg of milk-based substrate or 5-30 g per 1000 kg of milk-based substrate 10-30 g per 1000 kg of milk-based substrate.

Optionally, the use mentioned above regards the fermented milk product wherein said product is selected from a list consisting of: yogurt, fresh yogurt, fruit yogurt, yogurt beverage, yogurt product, stirred yogurt, drinking yogurt, post-pasteurized yogurt, post-pasteurized stirred yogurt, post-pasteurized drinking yogurt, skyr, Greek yogurt, fortified Greek yogurt, strained Greek yogurt, set yogurt, quark, Dahi, Labneh, buttermilk.

Optionally, the use mentioned above concerns a beta-galactosidase enzyme having an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

The disclosure also relates to the use of a beta-galactosidase, preferably a low pH stable beta-galactosidase or acidic beta-galactosidase, for maintaining sourness and/or sweetness of a fermented milk product constant over the shelf life of the fermented milk product as compared to sourness and/or sweetness of a fermented milk product yogurt deprived of beta-galactosidase,
wherein the beta-galactosidase enzyme that is added in a concentration, or an initial concentration, of less than 200 g per 1000 kg of milk-based substrate or less than 150 g per 1000 kg of milk-based substrate, preferably 5-140 g per 1000 kg of milk-based substrate or 5-90 g per 1000 kg of milk-based substrate or 10-70 g per 1000 kg of milk-based substrate or 30-50 g per 1000 kg of milk-based substrate, more preferably 5-35 g per 1000 kg of milk-based substrate or 5-30 g per 1000 kg of milk-based substrate 10-30 g per 1000 kg of milk-based substrate.

The disclosure also relates to the use of a beta-galactosidase, preferably a low pH stable beta-galactosidase or acidic beta-galactosidase, for maintaining post-acidification of a fermented milk product constant over the shelf life of the fermented milk product as compared to post-acidification of a fermented milk product yogurt deprived of beta-galactosidase,
wherein the beta-galactosidase enzyme that is added in a concentration, or an initial concentration, of less than 200 g per 1000 kg of milk-based substrate or less than 150 g per 1000 kg of milk-based substrate, preferably 5-140 g per 1000 kg of milk-based substrate or 5-90 g per 1000 kg of milk-based substrate or 10-70 g per 1000 kg of milk-based substrate or 30-50 g per 1000 kg of milk-based substrate, more preferably 5-35 g per 1000 kg of milk-based substrate or 5-30 g per 1000 kg of milk-based substrate 10-30 g per 1000 kg of milk-based substrate.

In any of the embodiments disclosed in the present section entitled “Uses” the beta-galactosidase enzyme that is or may be selected from a sequence having at least 80% sequence identity to SEQ ID NO: 1, 2, 3, 4, 5, 6, 7, 8, 9, 10, 11, 12, 13, 14, 15, 16, 17, 18, 19, 20 and/or 21, preferably at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21, more preferably least 85% or at least 90% or at least 91% or at least 92% or at least 93% or at least 94% or at least 95% or at least 96% or at least 97% or at least 98% or at least 99% or 100% sequence identity to SEQ ID NO: 21.

In any of the embodiments disclosed in the present section entitled “Uses” the beta-galactosidase enzyme has an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

### Compositions

Finally, the disclosure or invention also regards a composition comprising a lactic acid bacterium and a beta-galactosidase enzyme, such as a low pH stable beta-galactosidase or acidic beta-galactosidase, wherein the beta-galactosidase enzyme is present in the composition in an initial concentration such that when added to a milk-based substrate the concentration of the beta-galactosidase is less than 200 g of beta-galactosidase enzyme per 1000 kg of milk-based substrate or less than 150 g per 1000 kg of milk-based substrate, preferably 5-140 g per 1000 kg of milk-based substrate or 5-90 g per 1000 kg of milk-based substrate or 10-70 g per 1000 kg of milk-based substrate or 30-50 g per 1000 kg of milk-based substrate, more preferably 5-35 g per 1000 kg of milk-based substrate or 5-30 g per 1000 kg of milk-based substrate 10-30 g per 1000 kg of milk-based substrate.

Optionally, the composition comprises a lactic acid bacterium selected from a Streptococcus thermophilus and/or Lactobacillus delbrueckii subsp. bulgaricus, such as a lactic acid bacterium is selected from the group consisting of Streptococcus thermophilus strain deposited as DSM22932, DSM22935, DSM24090, DSM24023, DSM32502, DSM32503, DSM32504, DSM32505, DSM32506, DSM32507, DSM25850, DSM25851, DSM26722 and a strain having at least 95% sequence identity to DSM22932, DSM22935, DSM24090, DSM24023, DSM32502, DSM32503, DSM32504, DSM32505, DSM32506, DSM32507, DSM25850, DSM25851, DSM26722; and/or a lactic acid bacterium is selected from the group consisting of Lactobacillus delbrueckii subsp. bulgaricus deposited as DSM24074, DSM26420, DSM26421 and a strain having at least 95% sequence identity to DSM24074, DSM26420, DSM26421.

Optionally, the composition comprises a beta-galactosidase enzyme wherein the beta-galactosidase enzyme is selected from a sequence having at least 80% sequence identity to SEQ ID NO: 1, 2, 3, 4, 5, 6, 7, 8, 9, 10, 11, 12, 13, 14, 15, 16, 17, 18, 19, 20 and/or 21, preferably at least 85% or at least 90% or at least 91% or at least 92% or at least 93% or at least 94% or at least 95% or at least 96% or at least 97% or at least 98% or at least 99% or 100% sequence identity to SEQ ID NO: 1, 2, 3, 4, 5, 6, 7, 8, 9, 10, 11, 12, 13, 14, 15, 16, 17, 18, 19, 20 and/or 21, more preferably least 85% or at least 90% or at least 91% or at least 92% or at least 93% or at least 94% or at least 95% or at least 96% or at least 97% or at least 98% or at least 99% or 100% sequence identity to SEQ ID NO: 21.

Optionally, the composition comprises a beta-galactosidase enzyme having an activity of less than 1130 BLU/L_{milk-based substrate}, or less than 850 BLU/L_{milk-based substrate}, preferably 28-790 BLU/L_{milk-based substrate} or 28-510 BLU/L_{milk-based substrate} or 57-395 BLU/L_{milk-based substrate} or 170-280 BLU/L_{milk-based substrate}, more preferably 28-195 BLU/L_{milk-based substrate} or 28-170 BLU/L_{milk-based substrate} 57-170 BLU/L_{milk-based substrate}.

### Lactic Acid Bacteria as Starter Culture

Any lactic acid bacteria suitable for fermenting milk or milk-based substrate and produce, for example, an acidified milk product such as yogurt can be used in the present disclosure or invention. Therefore, lactic acid bacteria such as Streptococcus thermophilus and Lactobacillus delbrueckii ssp. bulgaricus can be used in the present disclosure or invention. For example, the lactic acid bacteria disclosed in WO2011092300, WO2018220104, WO2013160413, and/or WO2017103051 can be used herein. Further, a suitable starter culture can be a YoFlex® starter culture, a Premium® starter culture, and/or a Sweety® starter culture, commercialized by Chr. Hansen A/S. Examples of starter cultures may be F-DVS® YoFlex® Mild 1.0, F-DVS® YoFlex® Premium 1.0, F-DVS® YF-L922, F-DVS® YF-L904, F-DVS® YF-L706 or F-DVS® YF-L901, among others. However, there are many more examples well known to the skilled person as alternative starter cultures.

### Beta-Galactosidase Enzyme

Enzymes having lactase activity to be used in a method of the present disclosure or invention may be of animal, of plant or of microbial origin. Preferred lactases are obtained from microbial sources. The enzyme may, e.g., be derived from a strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Sclerotinia, e.g. S. libertiana; Torula; Torulopsis; Trichophyton, e.g. T. rubrum; Whetzelinia, e.g. W. sclerotiorum; Bacillus, e.g. B. coagulans, B. circulans, B. megaterium, B. novalis, B. subtilis, B. pumilus, B. stearothermophilus, B. thuringiensis; Bifidobacterium, e.g. B. longum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica.

Preferably, the lactase originates from a bacterium, e.g. from the family Bifidobacteriaceae, such as from the genus Bifidobacterium, such as from a strain of B. bifidum, B. animalis or B. longum. In a more preferred embodiment, the lactase originates from Bifidobacterium bifidum and may be NOLA® Fit from Chr. Hansen A/S or Saphera® from Novozymes A/S. Alternatively, a beta-galactosidase enzyme having at least 80% or at least 85% or at least 90% or at least 95% or at least 96% or at least 97% or at least 98% or at least 99% or 100% sequence identity to any of the sequences disclosed in WO2020079116, in particular SEQ ID NO: 1-20 and 22 in WO2020079116 (herein corresponding to SEQ ID NO: 1-21), can also be used.

### Fermented Milk Products

Fresh yoghurt generally has a shelf life of 28 days. The best selling time of fresh yogurt is influenced by the starter culture, the storage temperature of the yogurt once produced and if this is kept constant over time. In general, the best selling time of fresh yogurt is around day 21 after production, provided the storage temperature remained constant over time.

However, it is frequently observed a change in flavor of fresh yogurt around day 7, as a consequence of instable storage temperature or broken cold chain storage, which promotes production of lactic acid by lactic acid bacteria used as starter cultures. Thus, lactic acid is produced over the shelf life of fresh yogurt when the storage temperatures are instable. This is problematic not only due to the lactic acid production but also because the consumer will hardly buy two yogurts with the same identical flavor.

Lactases are known to hydrolyze lactose and produce glucose and galactose, both having a higher sweetness index than lactose. Thus, the hydrolysis of lactose results in an increase of sweetness perception. However, sweetness perception depends on the degree and amount of lactose hydrolysed. For example, if 200 g/ton of lactase (herein considered to be a high concentration of lactase) is added to the starter culture then, after fermentation, only a small fraction of the initial lactose can be used for lactic acid production since the majority of lactose is hydrolysed by lactase leading to a yogurt perceived by the consumer having a high sweetness.

A comparison between a fresh yogurt prepared with a high concentration of lactase versus a reference yoghurt without lactase (control) shows an excess of sweetness at the beginning of shelf life of the yogurt prepared with a high concentration of lactase versus the control. This results in a significant difference in flavor between the start and end of shelf life of the yogurt, which is not necessary desirable.

In contrast, by dosing a smaller amount of lactase (less than 200 g/ton of lactase is herein considered to be a small concentration of lactase), the majority of lactose will still remain in the yoghurt after fermentation. Comparing this yogurt with the control shows the flavor of both is similar, in particular with regard to the sourness and sweetness perceived by the consumuer. During the shelf life storage, both the control and the yoghurt prepared with a low concentration of lactase will continue to produce lactic acid; however, the yoghurt with the low concentration of lactase can still hydrolyze lactose slowly in parallel, this is helpful to add sweetness to yoghurt which can overcome the sourness generated by lactic acid bacteria.

Therefore, the amount or concentration of lactase added is relevant to the development of the proper flavor perception of fresh yogurt over the storage time, therefore keeping the yoghurt with a proper flavor for majority of the shelf life. In particular, it may be suggested that the minimum lactase dosage used leads to a lactose hydrolysis of about 75% shelf life at 25° C., wherein the about 75% shelf life corresponds to 21 days, or in other words when the fermented milk product has already been on the shelf for about 21 days and about 75% of its initial lactose has already been hydrolyzed. The maximum lactase dosage leads to a complete lactose hydrolysis after one week (D+7) after the end of fermentation and wherein the fermented milk product is kept at 25° C. Thus, the lactase dosage range for this purpose is adjustable according to the desired length of shelf life of the fermented milk product, such as yogurt. Finally, a milk-based substrate (milk or milk base) with significantly more than, for example, 4% w/w of lactose may need a readjustment on the concentration of lactase added, while a milk-based substrate having significantly less than, for example, 4% w/w of lactose may need a readjustment on the concentration of lactase added.

The present disclosure has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the subject-matter herein disclosed. These and other embodiments of the present disclosure will become readily apparent to those skilled in the art from the following detailed description of the preferred embodiments having reference to the whole description, the disclosure not being limited to any particular preferred embodiment(s) disclosed.

The use of the terms “a” and “an” and “the” and similar references in the context of describing the disclosure or invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1

Production of a Fermented Milk Product (Yogurt) Using a Lactase from Bifidobacterium bifidum

The composition of the milk base used was:

| Ingredients | Dosage | Brand | Nutritional information |
|---|---|---|---|
| Fresh milk | 93% (w/w) | Sanyuan | Protein: 2.79% |
| Sugar, such as | 3% (w/w) | Taigu | Fat: 3.16% |
| sucrose | | | Carbohydrates: 11.28% |
| Syrup HFCS55 | 4% (w/w) | Cargill | |

The ingredients of the milk base were mixed and allowed to re-hydrate. The milk base was then pasteurized at 95° C. for 5 minutes. The milk base was inoculated with 100 u/ton (units/ton) of F-DVS® YoFlex® Premium 1.0 (culture 1) or 100 u/T (units/ton) of F-DVS® YF-L907 (culture 2), both from Chr. Hansen A/S. Alternatively, other cultures well known to the skilled person may be used or Streptococcus thermophilus and/or Lactobacillus delbrueckii subsp. bulgaricus can also be used, such as Streptococcus thermophilus strains selected from DSM22932, DSM22935, DSM24090 and DSM24023 disclosed in WO2011092300, and/or DSM32502, DSM32503, DSM32504, DSM32505, DSM32506, DSM32507 disclosed in WO2018220104, and/or DSM25850, DSM25851, DSM 26722 disclosed in WO2013160413, and/or DSM3227 disclosed in WO2017103051 can also be used; and/or Lactobacillus delbrueckii subsp. bulgaricus strains selected from DSM24074, DSM26420, and/or DSM26421 disclosed in WO2011092300 and WO2013160413 can also be used.

Lactase was added to the milk base in the dosages indicated in Table 1. In this example, the lactase was added together with the culture; thus before fermentation. However, lactase could alternatively be added during or after fermentation. For example, it can be added after fermentation in case the fermented milk product is a post-pasteurized yogurt. A lactase suitable for the present disclosure or invention is a lactase from Bifidobacterium bifidum, for example, Saphera® from Novozymes A/S or NOLA® Fit 5500 from Chr. Hansen A/S, wherein 5500 corresponds to 5500 BLU/g of activity. Alternatively, a beta-galactosidase enzyme having at least 80% or at least 85% or at least 90% or at least 95% or at least 96% or at least 97% or at least 98% or at least 99% or 100% sequence identity to any of the sequences disclosed in WO2020079116, in particular SEQ ID NO: 1-20 and 22 in WO2020079116 (herein corresponding to SEQ ID NO: 1-21), can also be used.

The fermentations were carried out at 42° C. and in 3 L scale vats. Further details are given in Table 1. The yogurt was cooled in a post treatment unit at a cooling temperature of 25° C. at 2 bars and later stored at 6° C. Acidification was monitored with a PH meter. The pH at D+3 and at 4° C. was around 4.20 to 4.21 for samples wherein culture 1 was used, and around 4.29 to 4.34 for samples wherein culture 2 was used.

TABLE 1

| | | Lactase added to milk base | Fermentation | |
|---|---|---|---|---|
| Sample | Culture | substrate (g/ton) | time | End pH |
| 1 | 1 | 0 | 4 h 40 | 4.51 |
| 2 | | 200 | | |
| 3 | | 90 | | |
| 4 | | 70 | | |
| 5 | | 50 | | |
| 6 | | 30 | | |
| 7 | | 10 | | |
| 8 | | 5 | | |
| 9 | 2 | 0 | 4 h 30 | 4.50 |
| 10 | | 200 | | |
| 11 | | 90 | | |
| 12 | | 70 | | |
| 13 | | 50 | | |
| 14 | | 30 | | |
| 15 | | 10 | | |
| 16 | | 5 | | |

### Informal Sensory Evaluation

A sensory evaluation of the yogurt samples of example 1 was carried out with 7 blindfolded panelists. Samples at day 3 and stored at 4° C. were tested.

During sensory evaluation, all the panelists tasted each sample and used the online EyeQuestion® provided by Logic8 B.V. to rate the intensity of two attributes, namely sweetness and sourness, in the above samples (stirred yogurts).

After the sensory evaluation, Sensory Analysis-ANOVA was used with multiple comparison tests to analyze test data of sensory evaluation through the online EyeOpenR® provided by Qi Statistics and Logic8 B.V. to observe whether there were significant differences among all samples in sweetness and sourness.

The panel concluded that samples 2 and 10 were sweeter than the reference samples 1 and 9. No significant difference was detected for samples 3-4 and 11-12 versus reference samples 1 and 9, respectively. Finally, with regard to samples 5-8 and 13-16 no differences were detected versus the reference samples. These observations were independent of the starter culture used.

Thus, a concentration of less than 200 g/ton of lactase has no influence or significant influence on the yogurt taste at an early stage of the shelf life.

### pH Evaluation

The pH of the sample yogurts at day 3 and stored at 4° C. was also determined. Samples prepared with culture 1 and lactase (samples 2-8) had a pH which was, in average, 0.01 lower than samples prepared with culture 2 and lactase (samples 10-16). Thus, the pH difference is insignificant.

### Lactose Measurements

The concentration of lactose in w/w % in yogurt was measured by Lactosens® R from Chr. Hansen A/S on days 0, 7, 14 and 21 after fermentation and in yogurts kept at 25° C., wherein 25° C. mimics a disruption in the cold chain of the product. Day 0 after fermentation means immediately after the end of fermentation. In this example, a lactose content of 0.5% (w/w) is considered to be limit of 0 lactose, thus then the lactose content is <0.5% (w/w) it means that most or all lactose has been hydrolyzed. The values are presented in Table 2.

TABLE 2
Lactose content at 25° C. in w/w
%. EOF stands for end of fermentation.

| Sample | Milk base | EOF (D 0) | D + 7 | D + 14 | D + 21 |
|---|---|---|---|---|---|
| 1 | 4.1 | 3.60 | <0.03 | <0.03 | <0.03 |
| 2 | | 0.42 | <0.03 | <0.03 | <0.03 |
| 3 | | 1.84 | <0.03 | <0.03 | <0.03 |
| 4 | | 2.54 | 0.03 | <0.03 | <0.03 |
| 5 | | 2.88 | 0.18 | <0.18 | <0.18 |
| 6 | | 3.51 | 0.915 | 0.17 | <0.17 |
| 7 | | 3.93 | >0.92 | 1.48 | 0.92 |
| 8 | | 3.96 | >0.92 | >1.48 | >0.92 |
| 9 | | 3.80 | <0.15 | <0.15 | <0.15 |
| 10 | | 0.67 | <0.15 | <0.15 | <0.15 |
| 11 | | 2.24 | <0.15 | <0.15 | <0.15 |
| 12 | | 2.66 | <0.15 | <0.15 | <0.15 |
| 13 | | 3.45 | 0.15 | <0.15 | <0.15 |
| 14 | | 3.57 | 0.84 | 0.10 | <0.10 |
| 15 | | 4.01 | >1.45 | 1.45 | 1.08 |
| 16 | | 4.06 | >1.45 | >1.45 | >1.08 |

When the cold chain is breached, lactic acid bacteria start producing lactic acid which turns the yogurt sour. The present disclosure or invention shows that low concentrations of lactase, less than 200 g/ton of lactase, is responsible for counter-acting the sourness developed in yogurt over time, as the enzyme hydrolyzes lactose slowly and in a controlled manner, thereby generating glucose and galactose which are sweeter than lactose. In contrast, a concentration of lactase of 200 g/ton or more leads to a yogurt being quickly depleted of lactose (D+7) and therefore unable to maintain the proper flavor profile of yogurt over the shelf life when the cold chain is broken.

Thus, it is relevant to maintain a proper flavor of a fresh yogurt even if the cold chain is breached, and in particular to maintain an adequate flavor of yogurt at an early to middle shelf life (D+7, D+14). Thus, Table 2 shows that a dosage of 30 to 90 g/ton of lactase, preferably 30 to 70 g/ton of lactase, even more preferably 30 to 50 g/ton of lactase, contribute to a yogurt with the proper tasting profile at D+7 and D+14.

Identical results to those disclosed in Example 1 are expected for the remaining lactases herein disclosed.

### Example 2

Production of a Fermented Milk Product (Yogurt) Using a Lactase from Bifidobacterium bifidum

The composition of the milk base used was as follows:

| Ingredients | Dosage | Brand | Nutritional information |
|---|---|---|---|
| Fresh milk | 93% (w/w) | Sanyuan | Protein: 2.79% |
| Sugar, such as | 7% (w/w) | Taigu | Fat: 3.16% |
| sucrose | | | Carbohydrates: 11.28% |

The fermentations were carried out as explained in Example 1, with the exception that fewer lactase concentrations were used and that the samples were fermented at 42° C. and to an end pH of 4.50 for samples wherein both cultures 1 and 2 were used, respectively, to form yogurt.

TABLE 3

| | | Lactase added | Temperature | Fermentation | |
|---|---|---|---|---|---|
| Sample | Culture | (g/ton) | (° C.) | time | End pH |
| 1 | 1 | 0 | 42 | 4 h 15 | 4.50 |
| 2 | | 30 | | | |
| 3 | | 50 | | | |
| 4 | | 200 | | | |
| 5 | 2 | 0 | | 4 h 30 | |
| 6 | | 30 | | | |
| 7 | | 50 | | | |
| 8 | | 200 | | | |

### Informal Sensory Evaluation

The sensory evaluation of the yogurt samples of example 2 was carried out with 14 blindfolded panelists. The sensory evaluation at 4° C. and 25° C. was carried out as follows. The sensory evaluation method used was descriptive sensory evaluation. Before evaluation, all samples were stored at 13° C. for at least 1 hour and all samples were provided to the blindfolded panelists. The sensory evaluation and post-sensory evaluation was carried out as explained in example 1. The results of example 2 were identical to the ones of example 1.

### Lactose Measurements

The concentration of lactose in w/w % in yogurt was measured by Lactosens® R from Chr. Hansen A/S on days 0 (DO), D+7 and D+14 after fermentation and in yogurts kept at 25° C., wherein 25° C. mimics a disruption in the cold chain of the product. DO after fermentation means immediately after the end of fermentation ripening time of the yogurt. In this example, a lactose content of 0.5% (w/w) is considered to be limit of 0 lactose, thus then the lactose content is <0.5% (w/w) it means that most or all lactose has been hydrolyzed. The values are presented in Table 4.

TABLE 4
Lactose content at 25° C. in w/w
%. EOF stands for end of fermentation.

| Sample | Milk base | EOF (D 0) | D + 7 | D + 14 |
|---|---|---|---|---|
| 1 | 4.1 | 3.80 | <3.80 | <3.80 |
| 2 | | 3.12 | 0.55 | <0.08 |
| 3 | | 2.61 | 0.07 | <0.07 |
| 4 | | 0.51 | <0.51 | <0.51 |
| 5 | | 3.90 | <3.90 | <3.90 |
| 6 | | 3.09 | 0.72 | 0.1 |
| 7 | | 2.70 | 0.15 | <0.15 |
| 8 | | 0.39 | <0.39 | <0.39 |

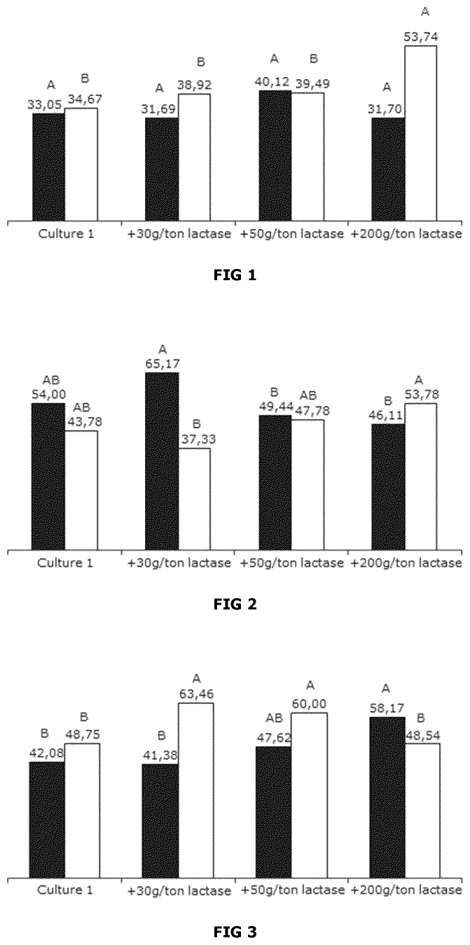

FIG. 1 shows the yogurt has the most balanced flavor at D+1 without the addition of lactase (control) or when lactase is added in low amounts, in particular wherein less than 200 g/ton of milk base (or milk-based substrate) is added. However, the yogurt sample with 200 g/ton of lactase, such as 200 g/ton of NOLA® Fit from Chr. Hansen A/S, has an excessive impact in the sweetness of the sample when compared to the sample without lactase. In contrast, low amounts of lactase, such as 30 g/ton or 50 g/ton, have a less significant impact in the yogurt tasting/flavor profile, and thus lead to a more constant tasting/flavor profile than when a high concentration of lactase is added (200 g lactase/ton milk or milk-base or milk-based substrate).

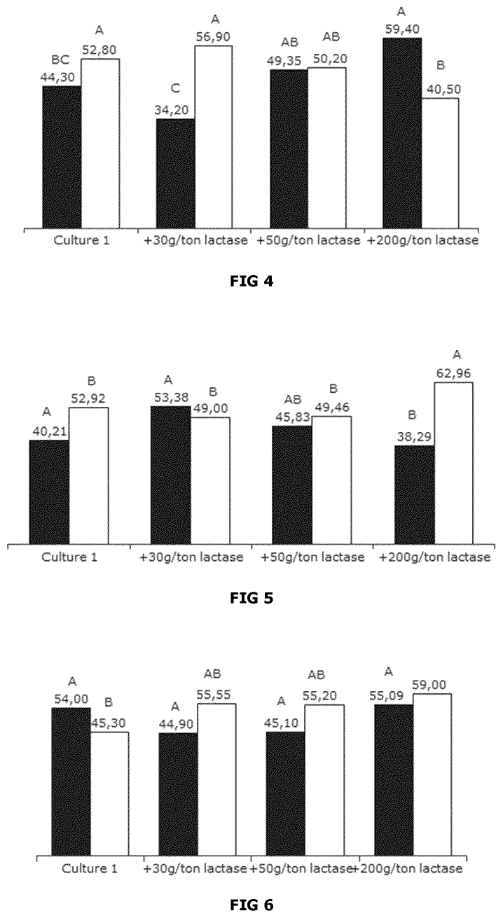

FIGS. 2-4 show that over the shelf life of the sample with 200 g/ton of lactase has a continuous lose of sweetness and increase sourness score, meaning that the tasting profile of this sample changes significantly in about 21 days after production. In contraste, low amounts of lactase are responsible for a steady increase in sweetness over the shelf life of the yogurt (D+7, D+14 and D+21).

FIGS. 5-7 show the yogurt profile when the cold chain is broken and the yogurt samples are subjected to undesired temperature, such as 25° C. At D+7 (FIG. 5), yogurt samples with 200 g/ton of lactase have more sweetness and less sourness than the control (only culture). In contrast, samples with low amounts of lactase, such as 30 g/ton or 50 g/ton of lactase, keep a similiar sensory profile as the control.

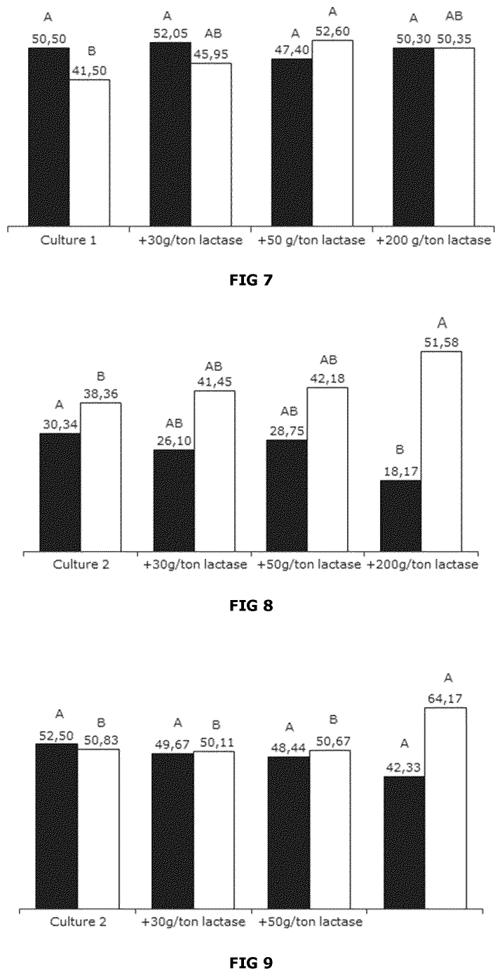

At D+14, D+21 (FIGS. 6-7), both the control and samples with 200 g/ton of lactase show an increase in sourness coupled to a decrease in sweetness. This means that the control is turning sour over the shelf life of yogurt when the cold chain is disrupted and that the samples with 200 g/ton is unable to maintain its initial sweetness. However, yogurt samples with 30 g/ton or 50 g/ton of lactase keep the sensory profile (balance of sourness and sweetness) similar as the initial premium1.0 (D+7) over shelf life (D+7, D+14, D+21).

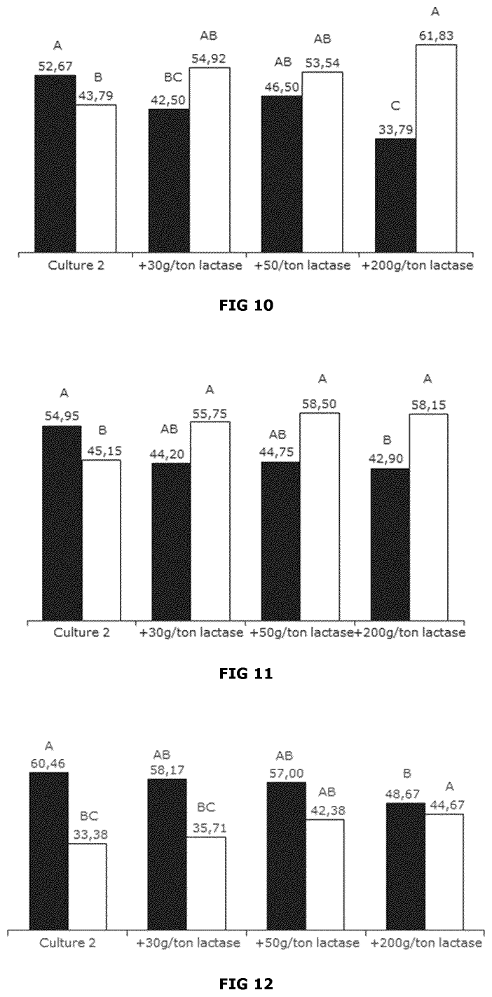

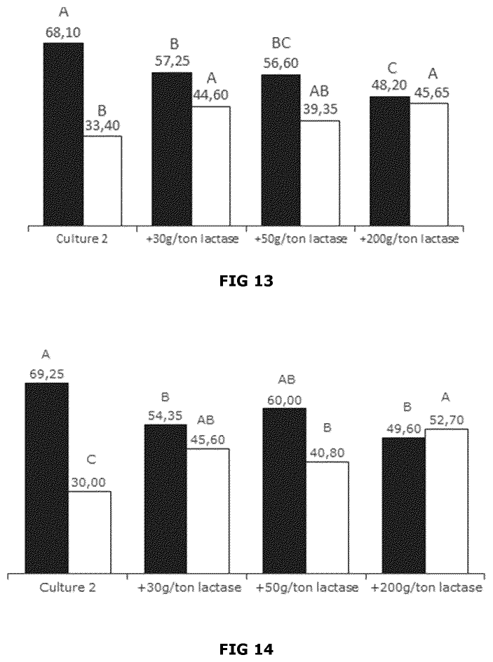

Similar results are obtained when a different culture was used (FIGS. 8-14) showing the balance of sourness and sweetness over time is a direct result of the addition of lactase in a concentration inferior to 200 g/ton and not of the culture used.

Identical results to those disclosed in Example 2 are expected for the remaining lactases herein disclosed.

### Example 3

Production of a Fermented Milk Product (Yogurt) Using a Lactase from Bifidobacterium bifidum

The composition of the milk base used was:

| Ingredients | Dosage | Brand | Nutritional information |
|---|---|---|---|
| Fresh milk | 89% (w/w) | Sanyuan | Protein: 2.67% |
| Sugar, such as | 2% (w/w) | Taigu | Fat: 3.03% |
| sucrose | | | Carbohydrates: 10.84% |
| Syrup HFCS55 | 5% (w/w) | Cargill | |
| Water | 4% (v/v) | Nestle | |

The fermentations were carried out as explained in Example 1, with the exception that only one lactase concentration was used (30 g/ton of milk base or milk-based substrate) and that the milk base (or milk-based substrate) was inoculated with two cultures, culture 2 as in Examples 1 and 2, and 20 u/ton of F-DVS® FreshQ®11 from Chr. Hansen A/S (culture 3).

TABLE 5

| | Cultures | Lactase added | Temperature | Fermentation | |
|---|---|---|---|---|---|
| Sample | (u/ton) | (g/ton) | (° C.) | time | End pH |
| 1 | 2 + 3 | 0 | 42 | 5 h | 4.34 |
| 2 | | 30 | | | 4.35 |

### Sensory Evaluation

The sensory evaluation of the yogurt samples of example 3 was carried out with blindfolded panelists. Samples at D+3, D+7 and D+14 and stored at 4° C. were tested. The sensory evaluation method used was descriptive sensory evaluation. Before evaluation, all samples were stored at 13° C. for at least 1 hour and all samples were provided to the blindfolded panelists. The sensory evaluation and post-sensory evaluation was carried out as explained in example 1.

The panel concluded that the yogurt sample with supplemented with lactase had, in the beginning of the shelf life, the same sensory profile as the reference. However, with the increase of the shelf life, the sample with lactase had more sweetness and less sourness compared with the reference.

### Lactose Measurements

The concentration of lactose in % in yogurt was measured by Lactosens® R from Chr. Hansen A/S on days 0, D+1, D+7, D+14, D+21 and D+28 after fermentation and in yogurts kept at 4, 12 and 25° C., wherein 12° C. and 25° C. mimic a disruption in the cold chain of the product. Day 0 after fermentation means immediately after the end of fermentation. In this example, a lactose content of 0.5% (w/w) is considered to be limit of 0 lactose, thus then the lactose content is <0.5% (w/w) it means that most or all lactose has been hydrolyzed. The values are presented in Table 6.

TABLE 6

| Sample | Milk base | D0 | D + 1 | D + 14 | D + 21 | D + 28 |
|---|---|---|---|---|---|---|
| | Lactose content at 4° C. in w/w % | | | | | |
| 1 | 4.4 | 3.60 | 3.40 | 3.00 | — | 3.30 |
| 2 | | 3.84 | 3.27 | 1.36 | 0.98 | 0.60 |
| | Lactose content at 12° C. in w/w % | | | | | |
| 1 | 4.4 | 3.60 | 3.20 | 3.10 | — | 3.10 |
| 2 | | 3.84 | 2.37 | 1.22 | 0.76 | 0.40 |
| | Lactose content at 25° C. in w/w % | | | | | |
| 1 | 4.4 | 3.60 | 3.30 | 3.10 | — | 3.10 |
| 2 | | 3.84 | 2.13 | 0.365 | 0.095 | 0.036 |

Example 3 demonstrates the different lactose hydrolyzing speed depending on the temperature. At a temperature of 4-6° C. (which corresponds to the normal temperature of a cold chain), the lactose residue is 0.6% on day 28. At 12° C., the lactose residue is 0.76% at day 21 and 0.4% at day 28. At 25° C., lactose residue is 0.365% at day 14. Table 6 shows that a higher the storage temperature leads to a faster lactose hydrolysis speed. It is also common that lactic acid bacteria post acidify faster when the storage temperature increases. The organoleptic properties of the yogurt are essentially maintained during its shelf life.

With sweet glucose and galactose accumulating in yoghurt, it is able to conquer the accumulated sourness. shelf life flavor changes are more moderate.

Furthermore, Table 7 shows how the post acidification avoided by the present disclosure or invention.

TABLE 7

| | D + 1 | D + 7 | D + 14 | D + 21 | D + 28 |
|---|---|---|---|---|---|
| 12° C. | | | | | |
| TA-sample 1 | 73.8 | 83.28 | 87.4 | 86.94 | 87.58 |
| TA-sample 2 | 73.5 | 83.1 | 87.46 | 86.6 | 86.54 |
| pH-sample 1 | 4.27 | 4.07 | 3.95 | 3.96 | 3.96 |
| pH-sample 2 | 4.29 | 4.07 | 3.94 | 3.96 | 3.97 |
| 25° C. | | | | | |
| TA-sample 1 | 78.62 | 97.38 | 100.5 | 99.88 | 100.78 |
| TA-sample 2 | 77.16 | 97.36 | 98.58 | 99.44 | 101 |
| pH-sample 1 | 4.17 | 3.88 | 3.81 | 3.81 | 3.82 |
| pH-sample 2 | 4.18 | 3.89 | 3.85 | 3.82 | 3.82 |

Similar results to the ones obtained in Examples 1-3 were also obtained when milk base was inoculated with 100 u/T of F-DVS® Sweety® Y-1 (culture 4) or 100 u/T of F-DVS® of Sweety® Y-3 (culture 5) and lactase was used in a concentration of 50 or 100 g/ton of milk base or milk-based substrate.

Identical results to those disclosed in Example 3 are expected for the remaining lactases herein disclosed.

### REFERENCES

WO2015193459, WO2015193449, WO2018130630, WO2011092300, WO2018220104, WO2013160413, WO2017103051, WO2020079116, WO2011092300, WO2013160413, WO2018220104

### SEQUENCE LISTING

SEQUENCELISTING

SEQ ID No 1
MRRNFEWPKLLTADGRGIAFGGDYNPDQWSEDIWDDDIRLMKQAGVNTVALAIFSWDRIQPTEDRWDFGWLDRIIDK
LGNAGIAVDLASATATAPLWLYESHPEVLPRDKYGHPVNAGSRQSWSPTSPVFKEYALTLCRKLAERYGTNPYVTAW
HMGNEYGWNNREDYSDNALDAFRAWCRRKYGTIGALNQAWGTTEWGQEMNGFDEVLIPRFMGADSMVNPGQKLDFER
FGNDMLLDFYKAERDAIAEICPDKPFTTNFMVSTDQCCMDYAAWAEEVNFVSNDHYFHEGESHLDELACSDALMDSL
ALGKPWYVMEHSTSAVQWKPLNTRKRKGETVRDSLAHVAMGADAINFFQWRASAFGAESFHSAMVPHAGEDTKLFRQ
VCELGASLHTLADAGVQGTELAHSDTAILFSAESEWATRSQTLPSMKLNHWHDVRDWYRAFLNAGSRADIVPLAYDW
SSYKTVVLPTVLILSAADTQRLADFAAAGGRVVVGYATGLIDEHFHTWLGGYPGAGDGLLRSMLGVRGEEFNILGAE
AEGEPGEIRLSSADDSAALDGTTTRLWQNDVNVTGEHAQVLATYAGEEADEWELDGTAAVTRNPYGSGEAYFVGCDL
DVADLTKLVRAYLAAPSQDNADVLHTVRESADATEDFYLPRGKETVELQGIEGEPVILFQTERGKKPGSYTVHRNGV
LVVRR
SEQ ID No 2
MNQRREHRWPRPLEGRRARIWYGGDYNPDQWPEEVWDEDVRLMVKAGVNLVSVGIFSWAKIEPREDMYDFGWLDRII
DKLGKAGIAVDLASATASPPMWLTQAHPEVLWKDYRGDVCQPGARQHWRPTSPVFCEYALKLCRAMAEHYKDNPYVV
AWHVGNEYGCHNRFDYSEDAERAFQDWCEERYGTIEAVNDAWGTAFWAQHLNDFSEIVPPRFIGDGNEMNPGKLLDF
KRFSSDALKSFYVAERDALAEITPEKPLTTNFMVSAGGSVLDYDDWGGEVDFVSNDHYFIPGEAHLDELAFSASLVD
GISRKDPWFLMEHSTSAVNWRPINYRKEPGQLVRDSLAHVAMGADAVCYFQWRQSRSGAEKFHSAMLPHAGEDSQTF
RDVCELGRDLGTLADEGLLGTKLAKSSVAIVFDYESEWASEHTATPTQNVHHIDEPLAWFRALADVGVTADVVPIRS
NWDEYDVAILPSVYILSEENTRRVRDYVANGGKLIATYYTGISDERDHVWLGGYPGSIRDVVGVRIEEFAPMGSDWP
GVPDHLDLDNGAVAHDIVDVIGSIGKDAKVLASFKDDPWTGMDGRPAIVSNPYGEGRSVYVGARLGRDGIARSLPMI
LETLGVEVKDSSDPDLLRIERVDESTGARFTFLFNRTKEPVSMLVEGRPVVMSLADCAGATVTINPNGVLVVKQ
SEQ ID No 3
MRRNFEWPKLLTADGRGIAFGGDYNPDQWSEDIWDDDIRLMKQAGVNTVALAIFSWDRIQPTEDRWDFGWLDRIIDK
LGNAGIAVDLASATATAPLWLYESHPEVLPRDKYGHPVNAGSRQSWSPTSPVFKEYALTLCRKLAERYGINPYVTAW
HMGNEYGWNNREDYSDNALDAFRAWCRRKYGTIGALNQAWGTTFWGQEMNGFDEVLIPRFMGADSMVNPGQKLDFER
FGNDMLLDFYKAERDAIAEICPDKPFTTNFMVSTDQCCMDYAAWAEEVNFVSNDHYFHEGKSHLNKLACSDALMDSL
ALGKPWYVMEHSTSAVQWKPLNTRKRKGETVRDSLAHVAMGADAINFFQWRASAFGAESFHSAMVPHAGEDTKLFRQ
VCELGASLHTLADAGVQGTELAHSDTAILFSAESEQATRSQTLPSMKLNHWHDVRDWYRAFLDAGSRADIVPLAYDW
SSYKTVVLPTVLILSAADTQRLADFAAAGGRVVIGYATGLIDEHFHTWLGGYPGAGDGLLRLMLGVRGEEFNILGAE
AEGEPSEIRLASADDSVAMDGSTTRLWQNDVNVTGEHAQVLATYAGEEADEWELDGTAAVTRNPYGSGEAYFVGCDL
DVADLTKLVRAYLAAPSQDNADVLHTVRESADATFDFYLPRGKETVELQGIEGEPVILFQTERGKKPGSYTVHRNGV
LVVRR
SEQ ID No 4
MTKTLSRFLYGGDYNPDQWTEETWPEDIKVFKKVDLNSATINIFSWAVLEPREGVYDFSKLDKIVQELSDANFDIVM
GTATAAMPAWMFKKYPDIARVDYQGRRHVFGQRHNFCPNSKNYQRLDSELVEKLAQHYADNSHIVVWHVNNEYGGNC
YCGNCQNAFRDWLRNKYKTLGALNKAWNMNVWSHTIYDWDEIVVPNELGDAWGPESSETIVAGLSIDYLRFQSESLQ
NLFKMEKAVIKKYDPETPVTTNFHSLPNKMIDYQKWAKDQDIISYDSYPTYDAPAYKPAFLYDLMRSLKHQPFMLME
SAPSQVNWQSYSPLKRPGQMAATELQAVAHGADTVQFFQLKQAVGGSEKFHSAIIAHSQRTDTRAFCELADLGQKLK
EAGPTILGSKTKAKVAIVFDWSNFWSYEYVDGITQDLNYVDSILDYYRQFYERNIPTDIIGVDDDFSNYDLVVAPVL
YMVKAGLAEKINSYVEKGGHLVTTYMSGMVDSTDNVYLGGYPGPLKDVTGIWVEESDAMVPGQKVRVTMDGKEYETN
LMCDLIHPNKAKVLASYADEFYTGTAAITENDYGKGKAWYVGTKLGHQGLTQLFNHIVLETGVESLVCDSHKLEVTK
RVTADGKELYFVLNMSNEERELPNKFADYEDILTGEKAKSSMKGWDVQVLTK
SEQ ID No 5
MKANIKWLDDPEVFRINQLPAHSDHPFYKDYREWQNHSSSFKQSLNGAWQFHFSKDPQSRPIDFYKRSFDSSSFDTI
PVPSEIELNGYAQNQYTNILYPWESKIYRKPAYTLGRGIKDGDFSQGKDNTVGSYLKHFDLNPALAGHDIHIQFEGV
ERAMYVYLNGHFIGYAEDSFTPSEFDLTPYIQAKDNILAVEVFKHSTASWLEDQDMFRFSGIFRSVELLALPRTHLM
DLDIKPTVVNDYHDGVFNAKLHFMGKTSGNVHVLIEDIDGKTLLNKKLPLKSTVEIENETFANVHLWDNHDPYLYQL
IIEVHDQDGKLVELIPYQFGFRKIEITKDHVVLLNGKRLIINGVNRHEWDAKRGRSITLADMKQDIATFKHNNINAV
RTCHYPNQIPWYYLCDQNGIYMMAENNLESHGTWQKLGQVEATSNVPGSIPEWREVVVDRARSNYETFKNHTAILFW
SLGNESYAGSNIAAMNKLYKDHDSSRLTHYEGVFHAPEFKKEISDLESCMYLPPKEAEEYLQNPKKPLVECEYMHDM
GTPDGGMGSYIKLIDKYPQYMGGFIWDFIDQALLVHDPVSGQDVLRYGGDEDDRHSDYEFSGDGLMFADRTPKPAMQ
EVRYYYGLHK
SEQ ID No. 6
MAYTNNLHVVYGEASLGVNGQDFAYLFSYERGGLESLKIKDKEWLYRTPTPTFWRATTDNDRGSGFNQKAAQWLGAD
MFTKCVGIHVQVDDHRFDELPVAPINNQFSNQEFAHEVKVAFDYETLTTPATKVKIIYNINDFGHMTITMHYFGKKG
LPPLPVIGMRFIMPTKAKSFDYTGLSGETYPDRMAGAERGTFHIDGLPVTKYLVPQENGMHMQTNELVITRNSTQNN
ADKDGDFSLKITQTKQPFNFSLLPYTAEELENATHIEELPLARRSVLVIAGAVRGVGGIDSWGSDVEEQYHIDPEQD
HEFSFTLN
SEQ ID No 7
MERNMSKRRKHSWPQPLKGAESRLWYGGDYNPDQWPEEVWDDDIRLMKKAGVNLVSVGIFSWAKIEPEEGKYDFDWL
DRAIDKLGKAGIAVDLASATASPPMWLTQAHPEVLWKDERGDTVWPGAREHWRPTSPVFREYALNLCRRMAEHYKGN
PYVVAWHVSNEYGCHNRFDYSDDAMRAFQKWCKKRYKTIDAVNEAWGTAFWAQHMNDFSEIIPPRYIGDGNFMNPGK
LLDYKRFSSDALKELYIAERDVLESITPGLPLTTNFMVSAGGSMLDYDDWGAEVDFVSNDHYFTPGEAHFDEVAYAA
SLMDGISRKEPWFQMEHSTSAVNWRPINYRAEPGSVVRDSLAQVAMGADAICYFQWRQSKAGAEKWHSSMVPHAGED
SQIFRDVCELGADLGRLSDEGLMGTKTVKSKVAVVFDYESQWATEYTANPTQQVDHWTEPLDWFRALADNGITADVV
PVRSDWDSYEIAVLPCVYLLSEETSRRVREFVANGGKLFVTYYTGLSDENDHIWLGGYPGSIRDVVGVRVEEFAPMG
NDMPGALDHLDLDNGTVAHDFADVITSTADTSTVLASYKAERWTGMNEVPAIVANGYGDGRTVYVGCRLGRQGLAKS
LPAMLGSMGLSDLAGDGRVLRVERADAAAASHFEFVFNRTHEPVTVDVEGEAIAASLAHVDDGRATIDPTGVVVLRR
SEQ ID No 8
MKRELKSKVFLHGGDYNPEQWLGEPEIINEDFALFKNAAINTVTVGIFSWAKLEPEEGKYDFAWLDDIFDRVEKMNG
YVILATPSGARPAWLARKYPEVLRTDFNNQKRGFGGRHNHCLTSPIYRKKVREINTKLAEHFGKRPSLILWHISNEY
SGECYCDLCQQAFRDWLKKKYRTLERLNHSWWNTFWSHTFSDWNQIHAPSPLSEMGNKGMNLDWKRFVSDQAISFID
NEVEPLRKITSEIPVTTNMMAGNPLMDPFTGYNYQEMAKHLDVISWDSYPLWGNDFQSTEKLGQNVGLIHDFFRSLK
HQNFMIMENTPSRVNWADIDRAKRPGMHQLASLQDIAHSSDSVLYFQLRASRGSAEMFHGAVIEHRHPEKTRVFHDV
KDVGHDLEKLESIYSTSYTKAKVGIVYDYNNIWALEDAEGYSKDKKIWQTIQSQYQYFYQNDIPVDFVSPNDNFTQY
KLLIDPMHFLMTKEYMDKLESFVKKCGYVVGTYISGVVDENGLAYMNEWPKQLQSIYGIEPLETDSLYPKQSNSIEF
AGHRYQAYDFCETIFKHDAKVLAKYTTDFYSGTPALTAHKCGEGKGYYIACRTDTDFLSAIYGQIVKELDLLPNLPI
KKETTKISLQVRENDDEKYLFVQNFSHEQQSILLKQKMKEMLSDEFEENKVIVKPYGTKIYQMN
SEQ ID No 9
MTQRRSYRWPQPLAGQQARIWYGGDYNPDQWPEEVWDDDVRLMKKAGVNLVSVGIFSWAKIETSEGVYDFDWLDRII
DKLGEAGIAVDLASATASPPMWLTQAHPEVLWKDYRGDVCQPGARQHWRPTSPVFREYALKLCRAMAEHYKGNPYVV
AWHVSNEYGCHNRFDYSEDAERAFRKWCEERYGTIDAVNDAWGTAFWAQRMNDFTEIVPPRFIGDGNFMNPGKLLDF
KRFSSDALKAFYVAERDALAEITPDLPLTTNFMVSAAGSVLDYDDWGREVDFVSNDHYFIPGEAHLDELAFSASLVD
GIARKDPWFLMEHSTSAVNWRPVNYRKEPGQLVRDSLAHVAMGADAVCYFQWRQSKAGAEKFHSAMVPHTGEDSAVF
RDVCELGADLNTLADNGLLGTKLAKSKVAVVFDYESEWATEHTATPTQKVHHVDEPLQWFRALADHGVTADVVPVSS
NWDEYEVVVLPSVYILSEETTRRVRDYVVNGGRLIVTYYTGLSDEKDHVWLGGYPGSIRDVVGVRVEEFMPMGDDFP
GVPDCLGLSNGAVAHDIADVIGSVDGTATVLETFRDDPWTGMDGAPAIVANTFGEGRSVYVGARLGRDGIAKSLPEI
FESLGMAETGENDSRVLRVEREGSDGSRFVFSFNRTHEAVQIPFEGKIVVSSFAEVSGENVSIKPNGVIVTKQ
SEQ ID No 10
MTQRRAYRWPQPLAGQQARIWYGGDYNPDQWPEEVWDDDVRLMKKAGVNLVSVGIFSWAKIETSEGVYDFDWLDRII
NKLGEAGIAVDLASATASPPMWLTQAHPEVLWKDYRGDVCQPGARQHWRPTSPVFREYALKLCRAMAEHYKGNPYVV
AWHVSNEYGCHNRFDYSEDAERAFRKWCEERYGTIDAVNDAWGTAFWAQRMNDFTEIVPPRFIGDGNFMNPGKLLDF
KRFSSDALKAFYVAERDALAEITPDLPLTTNFMVSAAGSVLDYDDWGREVDFVSNDHYFIPGEAHLDELAFSASLVD
GIARKDPWFLMEHSTSAVNWRPVNYRKEPGQLVRDSLAHVAMGADAVCYFQWRQSKAGAEKFHSAMVPHAGEDSAVF
RDVCELGADLNTLADNGLLGTKLAKSKVAVVFDYESEWASEHTATPTQKVHHVDEPLQWFRALADHGVTADVVPVRG
AWDDYEMVVLPSVYLLSEETTRRVRDYVVGGGRLVVTYYTGISDEKDHVWLGGYPGSIRDVVGVRVEEFMPMGDDEP
GVPDCLGLSNGAVAHDIADVIGSVDGTATVLETFKDDPWTGMDGAPAIVAHTFGEGRSVYVGARLGRDGIALSLPEI
LDSLGMAEAGGNDGRVLRVEREGADGSRFVFSFNRTHETVRVPVEGEVVVSSFAEVSGETISIKPNGVIVTKQ
SEQ ID No 11
MKRILNTNEFLHGGDYNPEQWWDEPDVINQDFALFKQAKINTVTVGIFSWAKLEPEEGNYDFSWLDSIFDRVEEMNG
HVVLATPSGARPAWLAQKYPEVLRTDNLGNKRGFGGRHNHCLTSPIYREKVREINTKLAEHFGQRKSLVLWHISNEY
SGECYCESCKNAFRDWLKNKYGNLDNLNHAWWNTFWSHTYNDWSQVNPPSPLGEMGNKGMNLDWKRFITDQTISFID
NEAAPLRKITPNVPVTTNMMAGNPLMDPFAGFDYQKVAKHLDFISWDSYPAWGNDNQTTAELGRNVGLVHDFFRSLK
HQNFLVMENTPSRVNWHSVDRAKRPGMHELASLQDVARGSQGVLYFQLRASRGSSEMFHGAVIEHLHPEQTRAFKDV
TTVGKDLENIRPIINTNYAKARVAIVFSYDSYWALQDAESYSKDKKIWQTIQKHYRYFYKHDIPVDFVSVEDDFSNY
DLLIDPMHFLMSKAYLKKLASYVKNGGRVVGTYISGVVDENDLAYMNEWPKELQDIYGVEPLETDVLYPGQSNTLNF
DGHEYKAHDYCETLINCRGKVLAKYASDFYQDTPAVVEHEYGAGKGYYLACRTDYDLLEKFYEKITANLIPEFPVKK
FSSNISIQVRENKDQKYYFVQNFSDKSEQIKVDGELEDLLEKKIDRGEVVLNPFGSKIYYKKGN
SEQ ID No 12
MLEPEEGKYDFSELDKVVKKLSDANFDIVIGTSTAAMPAWMFKKYPDVARVDYQGRRHVFGQRYNFCPNSKNYQRLA
GNLVEELAKHYQNNPNIVVWHVNNEYGGNCYCENCQHEFRKWLKDKYQTLDALNKAWNMNVWSHTIYDWDEIVVSNE
LGDAWGPEGSETIVAGLSIDYLRFQSESLQNLFKMEKQIIKKHDSEAPVTTNFHSLPNKMIDYQKWAKDQDIISYDS
YHTYDAPTYKPAFLYNLMRSLKHQPFMLMESAPSQVNWQPYSPLKRPGQMAATELQAVAHGADTVQFFQLKQAVGGS
EKFHSAVIAHSQRTDTRVFKELVDLGHKLKRAGSTILGSTINAKVGIVFDWSNFWSYEYVDGISQDMDYVDSILDYY
RQFYERNIPTDIISVDDDFSKYDLIVAPVLYMVKDGLAEKINNYVECGGNFVTTYMSGMVDSTDNVYLGGYPGPLKN
VTGIWVEESDAVVPGHTTTVSLKGKDYKAGFVCDLIHPEQAKVLAEYSNEFYAGTPAITENKYGQGKAWYVGTRLDH
TGLTQLFNHIVLESNIESLVCDGDKLEVTKRVTQDGQELYFVLNMSNEVRNLPQKFIGYQDILTDKKASDKLERWGV
QVLTK
SEQ ID No 13
MTTHRAFRWPSLLTESGRGIAFGGDYNPDQWPEETLDEDIRLMGEAGVNVVSLAIFSWDKIEPVEGAFTFEWLDHVI
DRLGRAGIAVDLASATAAAPLWLYESHPEVLPVDRYGHTVNAGSRQSWQPTSPVFKEYALRLCRKLAEHYKDNPYVT
AWHMGNEYGWNNRYDYSDNALAAFRTWCEAKYGTIDALNEAWGTAFWSQHVNSFDEVLLPRHMGGDAMVNPSQQLDY
ERFGNDMLLDFYKAERDAIEQICPDKPFTTNFMVSTDQCVMNYAKWADEVDFVSNDHYFHEGESHLDELACSDALMD
SLALGKPWYVMEHSTSAVQWKPLNTRKRAGELMRDSLAHVAMGADAICFFQWRQSKSGAEAFHSAMLPHAGADSKVF
RGVCELGKALKTLSDAGLQGTELERAGTAILFSAESEWATRSETLPSMKLNHWHDVRDWYRGFLDAGLRADVVPLAY
DWTGYKTIVLPTVLSLSDEDVLRIADFAKAGGTVIVGYAAGLIDEHFHIGLGGYPGAGNGLLRDMLGIRSEEFNILG
EEAEGEPSEISLSNGLTTRLWQNDVTSVAADTTVLASYAGESAADWELERTPAITSRPYGNGTAIYVGCDLNRHDIA
QLLKALGSRWQELSAQPTESGQTPTYPTTDPRILHTIRRSADGSTREDFYLNRSNQPVAINGVEGDPIIAHRCETDA
VGYTLNRNAILIAKTSC
SEQ ID No 14
MERKEFKWPQPLAGNKPRIWYGGDYNPDQWPEEVWDEDVALMQQAGVNLVSVAIFSWAKLEPEEGVYDFDWLDRVID
KLGKAGIAVDLASGTASPPMWMTQAHPEILWVDYRGDVCQPGARQHWRATSPVFLDYALSLCRKMAEHYKDNPYVVS
WHVSNEYGCHNRFDYSEDAERAFQKWCEKKYGTIDAVNDAWGTAFWAQRMNNFSEIIPPRFIGDGNFMNPGKLLDWK
RFSSDALLDFYKAERDALLEIAPKPQTTNFMVSAGGTGIDYDKWGYDVDFVSNDHYFTPGEAHFDELAYSASLCDGI
ARKNPWFLMEHSSSAVNWRPINYRVEPGELVRDSLAHLAMGSDAICYFQWRQSKAGAEKWHSSMVPHAGPDSQIFRD
VCELGADLNKLADEGLLSTKLVKSKVAVVFDYESQWVTEHTATPTQEVRHWTEPLAWFRALADNGLTADVVPVRGSW
DEYEAVVLPSLTILSEETTRRVREYVANGGKLFVTYYTGLVDDKDHVWLGGYPGSIRDVVGVRVEEFAPMGNDFPGA
MDHLDLDNGTVAHDFADVITSVADTAHVVASFKADKWTGFDGAPAITVNDFGDGKAAYVGARLGREGLAKSLPALLE
ELGIETSAEDDRGEVLRVERADETGENHFVFLFNRTHDVAVVDVEGEPLVASLAQVNESERTAAIQPNGVLVVKL
SEQ ID No 15
MTTRRTFRWPSLLTESGRGIAFGGDYNPDQWPEETLDEDIRLMVQAGVNTVALAIFSWDKIEPREGEFTFEWLDHVI
DKLGAASIAVDLASATATAPLWLYERHPEVLPIDRYGHVVNAGSRQSWQPTSPVLKEYALRLCRKLAEHYKDNPYVT
AWHMGNEYGWNNRYDYSDNALAAFRTWCEAKYGTVDALNEAWGTAFWSQHVNSFDEVLLPRHMGGDSMVNPPQQLDY
ERFGNDMLLDFYKAERDAIEEICPGKPFTTNFMVSTDQCTMDYAQWANEVDFVSNDHYFHEGESHLDELACSDALMD
SLALGKPWYVMEHSTSAVQWKPLNTRKRAGELMRDSLAHVAMGADAINFFQWRQSASGAEAFHSAMVPHAGSDTKLF
RGVCELGAALKTLSDAGVQDTELKRADTAILFSAESEWATRSETLPSMKLNHWHDVRDWYRGYLDAGARADVVPLAY
DWSGYQTIVLPTVIALSDEDTRRIADFAENGGTVIVGYATGLIDEHFHIGLGGYPGAGNGLLRDMLGIRSEEFNILG
EEAEDEPAEIGLSNGLTTRLWQNDVTSVAPDTRVLATYVGTAAADWELDGVPAITSHPHGQGAAIYVGCDLGRHDIT
HLLKELNTTAPSDERAPDQRPGGGEINAATTTAAATTHDPRILHTIRQSSDGTIRFDFYLNRSKQPVAVNGVEGDPI
IAHRCETDAVGYTLNRNAILIAKTSC
SEQ ID No 16
MMKKELPRFLYGGDYNPEQWPEETWDEDIKVFKQADINSATINVFSWALLEPQEGKYDFTKLDKIIKELTVADFDIV
LATSTAAMPAWMFKKYPDVARVDYQGRRHVFGARHNFCPSSKNYRRLAKNLVEQLAKRYGDNPHIVAWHVNNEYGGN
CYCEECQTEFQQWLKARYQTLDNLNHAWNMNVWSHTIHDWNEIVVPNELGDAWGPEGSETIVAGLSIDYLRFQSAQM
LDLFKMEKQIIEKYDPTTLVTTNFHSLPNKMIDYQQWASAQDIISYDSYPAYDAPIYQPAFLYDLMRSLKHQPFMLM
ESTPSQVNWQPYSPLKRPGQMAATELQAVAHGADTVQFFQLKQALGGSEKFHGAVISHANRTDTRVFKEVAKLGHDL
RKVGPVIKDSQTKARVALIFDWSNFWSFEYVDGITQDLKYVPIILDYYRQFYELNIPTDVISVDDDFRQYDLVVAPV
LYMVKGGLGKKITDYVANGGNFITSFMSGMVNESDNIYPGGYPGPLKDVMGLWVEESDAILPNKDVKLIMTTGDELT
GYLIADLIRLNGAHVLAEYASEFYAGTPAVTENTYSKGKAWYVGSRLDHASLRKIIMHIVDDVHLSALVKEPTELEI
TKRQNSAGQDIYFVLNMGKGKQPLPVEFQKGYRDLLTGDSPETMLDSWDVEILVQE
SEQ ID NO 17
MSNKLVKEKRVDQADLAWLTDPEVYEVNTIPPHSDHESFQSQEELEEGKSSLVQSLDGNWLIDYAENGQGPINFYAE
DFDDSNFKSVKVPGNLELQGFGQPQYVNIQYPWDGSEEIFPPQVPSKNPLASYVRYFDLDEALWDKEVSLKFAGAAT
AIYVWLNGHFVGYGEDSFTPSEFMVTKFLKKEGNRLAVALYKYSSASWLEDQDFWRLSGLFRSVTLEAKPLLHLEDL
KLTASLTDNYQKGKLEVEANIAYRLPNASFKLEVRDSEGDLVAEKVGPIRSEKLGFSLADLPVAAWSAEKPNLYQVR
LYLYQAGSLLEVSRQEVGFRNFELKDGIMYLNGQRIVFKGVNRHEFDSKLGRAITEADMIWDIKTMKQSNINAVRCS
HYPNQSLFYRLCDKYGLYVIDEANLESHGTWEKVGHEDPSFNVPGDDQHWLGASLSRVKNMMARDKNHASILIWSLG
NESYAGTVFAQMADYVRKADPTRVQHYEGVTHNRKFDDATQIESRMYAPAKEIEEYLTKKPAKPFISVEYAHAMGNS
VGDLAAYTALEKYPHYQGGFIWDWIDQGLEKDGHLLYGGDFDDRPTDYEFCGDGLVFADRTTSPKLANVKALYSNLK
LEVKDGQLFIKNDNLFTNSSAYYFLASLLVDGKLTYQSQPLTFGLEPGESGTFVLPWPEVEDEKGEIVYQVTAHLKE
DLPWADEGFTVAEAEEAVTKLPEFYPAGRPELVDSDFNLGLKGNGFRILFSKAKGWPVSIKYAGREYLKRLPEFTFW
RALTDNDRGAGYGYDLAKWENAGKYARLQDISYEIKENSALVKTTFTLPVALKGDLTITYEVDSLGKIAVTANFPGA
VENGLLPAFGLNFALPKELSDYRYYGLGPNESYADRLEGSYLGIYQGAVEKNFTPYLRPQEAGNRSKVRYYQLFDEE
GGLEFTANGADLNLSALPYSAAQIEAADHAFELTNNYTWVRALAAQMGVGGDDSWGQKVHPEFCLDAQEARQLKLVI
QPLLLK
SEQ ID No 18
MAHRRTFHWPSLLTESGRGIAFGGDYNPDQWPEDVWDDDIRLMKQAGVNTVALAIFSWDRIQPEKHRWEFGWLDCII
DKLGKAGIAVDLASATATAPLWLYEQHPEVLPHDKYGHPINAGSRQSWSPTSPVFKEYALTLCRKLAERYGTNPYVT
AWHMGNEYGWNNRYDYCDNALHAFRAWCERKYGTIEALNAAWGTTFWGQEMNGFDEVLIPRFMGADSMVNPGQKLDF
ERFGNDMLLDFYRAERDAIAEICPDKPFTTNFMVSTDQCCMDYADWANEVDFVSNDHYFHEGESHIDELFCSDALMD
SLALGRPWYVMEHSTSAVQWKDLNIRKRKGETVRDSVAHVAMGADAINFFQWRASAFGAESFHSAMVPHAGEHTKLY
RSVCELGAALKTLGDAGVQGSELVRSDTAILFSAESEWATRSETLPSKKLNHWHDVRDWYRAYLDAGTRADIVPLKY
DWSGYATVVLPTVLMLSAADTARLERFVRDGGTVVVGYASGLIDENFHTWLGGYPGAGDGMLRTMLGIRGEEFNILG
AQAEGEPSEIRLSNGMVTRLWQNDIAVDGADTEVLASYAGTQADEWELDGTAAITRNPYGKGMAYFVGCDLNVADLA
VFVGDHLTVGQACEAGDGADYDPTITLHTERASAEAIFDFYLPRGKNETVVSGISGEPVYRFQCDEGEAPGVYTIRR
NGVLVVKRYNRQ
SEQ ID No 19
MEAELKWLDDPEVFRVNQLPAHSDHRFYRDQEEAALEKSSYVQNLNGRWGFKFSKNPMERPVDFYKLDFDRNDFGEI
EVPSEIELSNFAQINYTNITMPWTGKIYRRPAYTLGDNKEEGSFSQGQDNTVGSYVRHFTLAEGLKNHDVHVVFEGV
ERAMYVWLNGHFIGYAEDSFTPSEFDLTPYLVDGDNLLAVEVYKHATSSWIEDQDMFRFSGIFRDVNLVAQPSIHVQ
DLKINARVADDMKTGSLGLVLKMVGQPGSVQVEVADQTGAAVLNRQLNADGNWTMAPVQLVGIHLWDNHHPYLYQLT
LTVRDATGRVVEVIPYQFGFRRVEIDQDKVLRLNGKRLIINGVNRHEWNCHRGRAVTIEDMHTDLGIFKENNINAVR
TSHYPDQIPWYYLCDREGIYMMAENNLESHATWQKFGQDEPSYNVPGSLPQWKEAVVDRARSNYEIFKNHTAILFWS
VGNESYAGEDILAMNNYYKEVDDTRPVHYEGVVHTKEYRDQISDFESWMYLPPKEVEAYLKKNPDKPFIECEYMHSM
GNSVGGMGSYIKLLDKYPQYCGGFIWDFVDQAIEVVDPVTGQKSMRYGGDEDDHHADNEFSGDGICFADRTPKPAMQ
EVKYYYGLHK
SEQ ID No 20
MDYTNKLHVVYDDNILGLDGKDFQYLESYEQGGPESFKIKGKEWLYRSPRPTFWRATTDNDRGNGFNVSSVQWLAAD
YVLPCQDIALQVDGKDKKLPLAPKTNRYSNQEFAKKVKITFTYQTQTVPATTVQVSYTVKASGKIKVNVHYTGAQLP
SLPVLGWRMIMPTPATSFDYEGLSGETYPDRMAGGIEGTYHVEGLPVTPYLVPQENGMHMANKWVQITRATTLNNAD
PDAAPFRLKFEAPKKGKLNFSCLPYTSAELENATHPEELPAAHRTVLVIAGEVRGVGGIDSWGADVEEKYHIDATVD
HDFSFKIVPELN
SEQ ID No 21
MVEDATRSDSTTQMSSTPEVVYSSAVDSKQNRTSDFDANWKFMLSDSVQAQDPAFDDSAWQQVDLPHDYSITQKYSQ
SNEAESAYLPGGTGWYRKSFTIDRDLAGKRIAINFDGVYMNATVWFNGVKLGTHPYGYSPFSFDLTGNAKFGGENTI
VVKVENRLPSSRWYSGSGIYRDVTLTVTDGVHVGNNGVAIKTPSLATQNGGNVTMNLTTKVANDTEAAANITLKQTV
FPKGGKTDAAIGTVTTASKSIAAGASADVISTITAASPKLWSIKNPNLYTVRTEVLNGDTVLDTYDTEYGFRWTGFD
ATSGFSLNGEKVKLKGVSMHHDQGSLGAVANRRAIERQVEILQKMGVNSIRTTHNPAAKALIDVCNEKGVLVVEEVF
DMWNRSKNGNTEDYGKWFGQTIAGDNAVLGGDKDETWAKFDLISTINRDRNAPSVIMWSLGNEMMEGISGSVSDFPA
TSAKLVAWTKAADSTRPMTYGDNKIKANWNESNTMGDNLTANGGVVGTNYSDGANYDKIRTTHPSWAIYGSETASAI
NSRGIYNRTTGGAQSSDKQLTSYDNSAVGWGAVASSAWYDVVQRDFVAGTYVWTGFDYLGEPTPWNGTGSGAVGSWP
SPKNSYFGIVDTAGFPKDTYYFYQSQWNDDVHTLHILPAWNENVVAKGSGNKVPVVVYTDAAKVKLYFTPKGSTEKR
LIGEKSFTKKTTAAGYTYQVYEGTDKDSTAHKNMYLTWNVPWAEGTISAEAYDENNRLIPEGSTEGNASVTTTGKAA
KLKADADRKTITADGKDLSYIEVDVTDANGHIVPDAANRVTFDVKGAGKLVGVDNGSSPDHDSYQADNRKAFSGKVL
AIVQSTKEAGEITVTAKADGLQSSTVKIATTAVPGTSTEKTVRSFYYSRNYYVKTGNKPILPSDVEVRYSDGTSDRQ
NVTWDAVSDDQIAKAGSFSVAGTVAGQKISVRVTMIDEIGALLNYSASTPVGTPAVLPGSRPAVLPDGTVTSANFAV
HWTKPADTVYNTAGTVKVPGTATVFGKEFKVTATIRVQRSQVTIGSSVSGNALRLTQNIPADKQSDILDAIKDGSTT
VDANTGGGANPSAWTNWAYSKAGHNTAEITFEYATEQQLGQIVMYFFRDSNAVRFPDAGKTKIQISADGKNWTDLAA
TETIAAQESSDRVKPYTYDFAPVGATFVKVTVTNADTTTPSGVVCAGLTEIELKTATSKFVTNTSAALSSLTVNGTK
VSDSVLAAGSYNTPAIIADVKAEGEGNASVTVLPAHDNVIRVITESEDHVTRKTFTINLGTEQEFPADSDERD

1. A method for producing a fermented milk product comprising the following steps:
providing a milk-based substrate comprising lactose;
fermenting the milk-based substrate with a lactic acid bacterium until a pH below 5 is reached;
adding a beta-galactosidase enzyme;
wherein the beta-galactosidase enzyme is present in a concentration of less than 200 g beta-galactosidase enzyme/1000 kg of milk-based substrate.

2. The method according to claim 1, wherein the beta-galactosidase enzyme is present in an initial concentration of less than 150 g/1000 kg of milk-based substrate.

3. The method according to claim 1, wherein the beta-galactosidase enzyme is selected from a sequence having at least 80% sequence identity to SEQ ID NO: 1, 2, 3, 4, 5, 6, 7, 8, 9, 10, 11, 12, 13, 14, 15, 16, 17, 18, 19, 20 or 21

4. The method according to claim 1, wherein the lactose concentration of the fermented milk product after the end of fermentation is more than 0.42 mg_{lactose}/g_{fermented milk product}.

5. The method according to claim 1, wherein the milk-based substrate has at least 1% w_{lactose}/w_{milk-based substrate}.

6. The method according to claim 1, wherein the step of adding the beta-galactosidase enzyme is carried out before, during or after the fermenting step.

7. The method according to claim 1, wherein the lactic acid bacterium is from the species Streptococcus thermophilus or Lactobacillus delbrueckii subsp. bulgaricus.

8. The method according to claim 1, wherein the lactic acid bacterium is selected from the group consisting of:
a Streptococcus thermophilus strain having at least 95% sequence identity to DSM22932, DSM22935, DSM24090, DSM24023, DSM32502, DSM32503, DSM32504, DSM32505, DSM32506, DSM32507, DSM25850, DSM25851, and DSM26722; or
wherein the lactic acid bacterium is selected from the group consisting of: a Lactobacillus delbrueckii subsp. bulgaricus having at least 95% sequence identity to DSM24074, DSM26420, and DSM26421.

9. The method according to claim 1, wherein the fermented milk product is yogurt, fresh yogurt, fruit yogurt, yogurt beverage, yogurt product, stirred yogurt, drinking yogurt, post-pasteurized yogurt, post-pasteurized stirred yogurt, post-pasteurized drinking yogurt, skyr, Greek yogurt, fortified Greek yogurt, strained Greek yogurt, set yogurt, quark, Dahi, Labneh, or buttermilk.

10. The method according to claim 1, wherein the beta-galactosidase enzyme has an activity of less than 1130 BLU/L_{milk-based substrate}.

11-14. (canceled)

15. A composition comprising a lactic acid bacterium and a beta-galactosidase enzyme, wherein the beta-galactosidase enzyme is present in the composition in a concentration, such that when added to a milk-based substrate the concentration of the beta-galactosidase enzyme is less than 200 g of beta-galactosidase enzyme per 1000 kg of milk-based substrate,
wherein the lactic acid bacterium is selected from the group consisting of: a Streptococcus thermophilus strain having at least 95% sequence identity to DSM22932, DSM22935, DSM24090, DSM24023, DSM32502, DSM32503, DSM32504, DSM32505, DSM32506, DSM32507, DSM25850, DSM25851, and DSM26722; or
wherein the lactic acid bacterium is selected from the group consisting of: a Lactobacillus delbrueckii subsp. bulgaricus having at least 95% sequence identity to DSM24074, DSM26420, and DSM26421.

16. The method of claim 1, wherein the beta-galactosidase enzyme is a low pH stable beta-galactosidase.

17. The method of claim 1, wherein the beta-galactosidase enzyme is present in an initial concentration of 5-140 g/1000 kg of milk-based substrate.

18. The method of claim 1, wherein the beta-galactosidase enzyme is present in an initial concentration of 10-70 g/1000 kg of milk-based substrate.

19. The method of claim 1, wherein the beta-galactosidase enzyme is selected from a sequence having at least 90% sequence identity to SEQ ID NO: 1, 2, 3, 4, 5, 6, 7, 8, 9, 10, 11, 12, 13, 14, 15, 16, 17, 18, 19, 20 or 21.

20. The method of claim 1, wherein the beta-galactosidase enzyme is selected from a sequence having at least 95% sequence identity to SEQ ID NO: 1, 2, 3, 4, 5, 6, 7, 8, 9, 10, 11, 12, 13, 14, 15, 16, 17, 18, 19, 20 or 21.

21. The method of claim 1, wherein the lactose concentration of the fermented milk product after the end of fermentation is more than 0.67 mg_{lactose}/g_{fermented milk product}.

22. The method of claim 1, wherein the milk-based substrate has 4-40% w_{lactose}/w_{milk-based substrate}.

23. The method of claim 1, wherein the lactic acid bacterium is selected from the group consisting of: a Streptococcus thermophilus strain deposited as DSM22932, DSM22935, DSM24090, DSM24023, DSM32502, DSM32503, DSM32504, DSM32505, DSM32506, DSM32507, DSM25850, DSM25851 and DSM26722.

24. The method according to claim 1, wherein the beta-galactosidase enzyme has an activity of less than 850 BLU/L_{milk-based substrate}.


SEQUENCE LISTING (21 sequences)

SEQ ID NO: 1 - protein, 698 aa, Bifidobacterium adolescentis
MRRNFEWPKLLTADGRGIAFGGDYNPDQWSEDIWDDDIRLMKQAGVNTVALAIFSWDRIQPTEDRWDFGWLDRIIDKLGNAGIAVDLASATATAPLWLYESHPEVLPRDKYGHPVNAGSRQSWSPTSPVFKEYALTLCRKLAERYGTNPYVTAWHMGNEYGWNNREDYSDNALDAFRAWCRRKYGTIGALNQAWGTTFWGQEMNGFDEVLIPRFMGADSMVNPGQKLDFERFGNDMLLDFYKAERDAIAEICPDKPFTTNFMVSTDQCCMDYAAWAEEVNFVSNDHYFHEGESHLDELACSDALMDSLALGKPWYVMEHSTSAVQWKPLNTRKRKGETVRDSLAHVAMGADAINFFQWRASAFGAESFHSAMVPHAGEDTKLFRQVCELGASLHTLADAGVQGTELAHSDTAILFSAESEWATRSQTLPSMKLNHWHDVRDWYRAFLNAGSRADIVPLAYDWSSYKTVVLPTVLILSAADTQRLADFAAAGGRVVVGYATGLIDEHFHTWLGGYPGAGDGLLRSMLGVRGEEFNILGAEAEGEPGEIRLSSADDSAALDGTTTRLWQNDVNVTGEHAQVLATYAGEEADEWELDGTAAVTRNPYGSGEAYFVGCDLDVADLTKLVRAYLAAPSQDNADVLHTVRESADATFDFYLPRGKETVELQGIEGEPVILFQTERGKKPGSYTVHRNGVLVVRR

SEQ ID NO: 2 - protein, 690 aa, Bifidobacterium adolescentis
MNQRREHRWPRPLEGRRARIWYGGDYNPDQWPEEVWDEDVRLMVKAGVNLVSVGIFSWAKIEPREDMYDFGWLDRIIDKLGKAGIAVDLASATASPPMWLTQAHPEVLWKDYRGDVCQPGARQHWRPTSPVFCEYALKLCRAMAEHYKDNPYVVAWHVGNEYGCHNRFDYSEDAERAFQDWCEERYGTIEAVNDAWGTAFWAQHLNDFSEIVPPRFIGDGNFMNPGKLLDFKRFSSDALKSFYVAERDALAEITPEKPLTTNFMVSAGGSVLDYDDWGGEVDFVSNDHYFIPGEAHLDELAFSASLVDGISRKDPWFLMEHSTSAVNWRPINYRKEPGQLVRDSLAHVAMGADAVCYFQWRQSRSGAEKFHSAMLPHAGEDSQTFRDVCELGRDLGTLADEGLLGTKLAKSSVAIVFDYESEWASEHTATPTQNVHHIDEPLAWFRALADVGVTADVVPIRSNWDEYDVAILPSVYILSEENTRRVRDYVANGGKLIATYYTGISDERDHVWLGGYPGSIRDVVGVRIEEFAPMGSDWPGVPDHLDLDNGAVAHDIVDVIGSIGKDAKVLASFKDDPWTGMDGRPAIVSNPYGEGRSVYVGARLGRDGIARSLPMILETLGVEVKDSSDPDLLRIERVDESTGARFTFLFNRTKEPVSMLVEGRPVVMSLADCAGATVTINPNGVLVVKQ

SEQ ID NO: 3 - protein, 698 aa, Bifidobacterium adolescentis
MRRNFEWPKLLTADGRGIAFGGDYNPDQWSEDIWDDDIRLMKQAGVNTVALAIFSWDRIQPTEDRWDFGWLDRIIDKLGNAGIAVDLASATATAPLWLYESHPEVLPRDKYGHPVNAGSRQSWSPTSPVFKEYALTLCRKLAERYGTNPYVTAWHMGNEYGWNNREDYSDNALDAFRAWCRRKYGTIGALNQAWGTTFWGQEMNGFDEVLIPRFMGADSMVNPGQKLDFERFGNDMLLDFYKAERDAIAEICPDKPFTTNFMVSTDQCCMDYAAWAEEVNFVSNDHYFHEGKSHLNKLACSDALMDSLALGKPWYVMEHSTSAVQWKPLNTRKRKGETVRDSLAHVAMGADAINFFQWRASAFGAESFHSAMVPHAGEDTKLFRQVCELGASLHTLADAGVQGTELAHSDTAILFSAESEQATRSQTLPSMKLNHWHDVRDWYRAFLDAGSRADIVPLAYDWSSYKTVVLPTVLILSAADTQRLADFAAAGGRVVIGYATGLIDEHFHTWLGGYPGAGDGLLRLMLGVRGEEFNILGAEAEGEPSEIRLASADDSVAMDGSTTRLWQNDVNVTGEHAQVLATYAGEEADEWELDGTAAVTRNPYGSGEAYFVGCDLDVADLTKLVRAYLAAPSQDNADVLHTVRESADATFDFYLPRGKETVELQGIEGEPVILFQTERGKKPGSYTVHRNGVLVVRR

SEQ ID NO: 4 - protein, 668 aa, Lactobacillus amylovorus
MTKTLSRFLYGGDYNPDQWTEETWPEDIKVFKKVDLNSATINIFSWAVLEPREGVYDFSKLDKIVQELSDANFDIVMGTATAAMPAWMFKKYPDIARVDYQGRRHVFGQRHNFCPNSKNYQRLDSELVEKLAQHYADNSHIVVWHVNNEYGGNCYCGNCQNAFRDWLRNKYKTLGALNKAWNMNVWSHTIYDWDEIVVPNELGDAWGPESSETIVAGLSIDYLRFQSESLQNLFKMEKAVIKKYDPETPVTTNFHSLPNKMIDYQKWAKDQDIISYDSYPTYDAPAYKPAFLYDLMRSLKHQPFMLMESAPSQVNWQSYSPLKRPGQMAATELQAVAHGADTVQFFQLKQAVGGSEKFHSAIIAHSQRTDTRAFCELADLGQKLKEAGPTILGSKTKAKVAIVFDWSNFWSYEYVDGITQDLNYVDSILDYYRQFYERNIPTDIIGVDDDFSNYDLVVAPVLYMVKAGLAEKINSYVEKGGHLVTTYMSGMVDSTDNVYLGGYPGPLKDVTGIWVEESDAMVPGQKVRVTMDGKEYETNLMCDLIHPNKAKVLASYADEFYTGTAAITENDYGKGKAWYVGTKLGHQGLTQLFNHIVLETGVESLVCDSHKLEVTKRVTADGKELYFVLNMSNEERELPNKFADYEDILTGEKAKSSMKGWDVQVLTK

SEQ ID NO: 5 - protein, 626 aa, Lactobacillus amylovorus
MKANIKWLDDPEVFRINQLPAHSDHPFYKDYREWQNHSSSFKQSLNGAWQFHFSKDPQSRPIDFYKRSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGTPDGGMGSYIKLIDKYPQYMGGFIWDFIDQALLVHDPVSGQDVLRYGGDFDDRHSDYEFSGDGLMFADRTPKPAMQEVRYYYGLHK

SEQ ID NO: 6 - protein, 316 aa, Lactobacillus amylovorus
MAYTNNLHVVYGEASLGVNGQDFAYLFSYERGGLESLKIKDKEWLYRTPTPTFWRATTDNDRGSGFNQKAAQWLGADMFTKCVGIHVQVDDHRFDELPVAPINNQFSNQEFAHEVKVAFDYETLTTPATKVKIIYNINDFGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 7 - protein, 693 aa, Bifidobacterium bifidum
MERNMSKRRKHSWPQPLKGAESRLWYGGDYNPDQWPEEVWDDDIRLMKKAGVNLVSVGIFSWAKIEPEEGKYDFDWLDRAIDKLGKAGIAVDLASATASPPMWLTQAHPEVLWKDERGDTVWPGAREHWRPTSPVFREYALNLCRRMAEHYKGNPYVVAWHVSNEYGCHNRFDYSDDAMRAFQKWCKKRYKTIDAVNEAWGTAFWAQHMNDFSEIIPPRYIGDGNFMNPGKLLDYKRFSSDALKELYIAERDVLESITPGLPLTTNFMVSAGGSMLDYDDWGAEVDFVSNDHYFTPGEAHFDEVAYAASLMDGISRKEPWFQMEHSTSAVNWRPINYRAEPGSVVRDSLAQVAMGADAICYFQWRQSKAGAEKWHSSMVPHAGEDSQIFRDVCELGADLGRLSDEGLMGTKTVKSKVAVVFDYESQWATEYTANPTQQVDHWTEPLDWFRALADNGITADVVPVRSDWDSYEIAVLPCVYLLSEETSRRVREFVANGGKLFVTYYTGLSDENDHIWLGGYPGSIRDVVGVRVEEFAPMGNDMPGALDHLDLDNGTVAHDFADVITSTADTSTVLASYKAERWTGMNEVPAIVANGYGDGRTVYVGCRLGRQGLAKSLPAMLGSMGLSDLAGDGRVLRVERADAAAASHFEFVFNRTHEPVTVDVEGEAIAASLAHVDDGRATIDPTGVVVLRR

SEQ ID NO: 8 - protein, 680 aa, Lactobacillus brevis
MKRELKSKVFLHGGDYNPEQWLGEPEIINEDFALFKNAAINTVTVGIFSWAKLEPEEGKYDFAWLDDIFDRVEKMNGYVILATPSGARPAWLARKYPEVLRTDFNNQKRGFGGRHNHCLTSPIYRKKVREINTKLAEHFGKRPSLILWHISNEYSGECYCDLCQQAFRDWLKKKYRTLERLNHSWWNTFWSHTFSDWNQIHAPSPLSEMGNKGMNLDWKRFVSDQAISFIDNEVEPLRKITSEIPVTTNMMAGNPLMDPFTGYNYQEMAKHLDVISWDSYPLWGNDFQSTEKLGQNVGLIHDFFRSLKHQNFMIMENTPSRVNWADIDRAKRPGMHQLASLQDIAHSSDSVLYFQLRASRGSAEMFHGAVIEHRHPEKTRVFHDVKDVGHDLEKLESIYSTSYTKAKVGIVYDYNNIWALEDAEGYSKDKKIWQTIQSQYQYFYQNDIPVDFVSPNDNFTQYKLLIDPMHFLMTKEYMDKLESFVKKCGYVVGTYISGVVDENGLAYMNEWPKQLQSIYGIEPLETDSLYPKQSNSIEFAGHRYQAYDFCETIFKHDAKVLAKYTTDFYSGTPALTAHKCGEGKGYYIACRTDTDFLSAIYGQIVKELDLLPNLPIKKETTKISLQVRENDDEKYLFVQNFSHEQQSILLKQKMKEMLSDEFEENKVIVKPYGTKIYQMN

SEQ ID NO: 9 - protein, 689 aa, Bifidobacterium catenulatum
MTQRRSYRWPQPLAGQQARIWYGGDYNPDQWPEEVWDDDVRLMKKAGVNLVSVGIFSWAKIETSEGVYDFDWLDRIIDKLGEAGIAVDLASATASPPMWLTQAHPEVLWKDYRGDVCQPGARQHWRPTSPVFREYALKLCRAMAEHYKGNPYVVAWHVSNEYGCHNRFDYSEDAERAFRKWCEERYGTIDAVNDAWGTAFWAQRMNDFTEIVPPRFIGDGNFMNPGKLLDFKRFSSDALKAFYVAERDALAEITPDLPLTTNFMVSAAGSVLDYDDWGREVDFVSNDHYFIPGEAHLDELAFSASLVDGIARKDPWFLMEHSTSAVNWRPVNYRKEPGQLVRDSLAHVAMGADAVCYFQWRQSKAGAEKFHSAMVPHTGEDSAVFRDVCELGADLNTLADNGLLGTKLAKSKVAVVFDYESEWATEHTATPTQKVHHVDEPLQWFRALADHGVTADVVPVSSNWDEYEVVVLPSVYILSEETTRRVRDYVVNGGRLIVTYYTGLSDEKDHVWLGGYPGSIRDVVGVRVEEFMPMGDDFPGVPDCLGLSNGAVAHDIADVIGSVDGTATVLETFRDDPWTGMDGAPAIVANTFGEGRSVYVGARLGRDGIAKSLPEIFESLGMAETGENDSRVLRVEREGSDGSRFVFSFNRTHEAVQIPFEGKIVVSSFAEVSGENVSIKPNGVIVTKQ

SEQ ID NO: 10 - protein, 689 aa, Bifidobacterium pseudocatenulatum
MTQRRAYRWPQPLAGQQARIWYGGDYNPDQWPEEVWDDDVRLMKKAGVNLVSVGIFSWAKIETSEGVYDFDWLDRIINKLGEAGIAVDLASATASPPMWLTQAHPEVLWKDYRGDVCQPGARQHWRPTSPVFREYALKLCRAMAEHYKGNPYVVAWHVSNEYGCHNRFDYSEDAERAFRKWCEERYGTIDAVNDAWGTAFWAQRMNDFTEIVPPRFIGDGNFMNPGKLLDFKRFSSDALKAFYVAERDALAEITPDLPLTTNFMVSAAGSVLDYDDWGREVDFVSNDHYFIPGEAHLDELAFSASLVDGIARKDPWFLMEHSTSAVNWRPVNYRKEPGQLVRDSLAHVAMGADAVCYFQWRQSKAGAEKFHSAMVPHAGEDSAVFRDVCELGADLNTLADNGLLGTKLAKSKVAVVFDYESEWASEHTATPTQKVHHVDEPLQWFRALADHGVTADVVPVRGAWDDYEMVVLPSVYLLSEETTRRVRDYVVGGGRLVVTYYTGISDEKDHVWLGGYPGSIRDVVGVRVEEFMPMGDDFPGVPDCLGLSNGAVAHDIADVIGSVDGTATVLETFKDDPWTGMDGAPAIVAHTFGEGRSVYVGARLGRDGIALSLPEILDSLGMAEAGGNDGRVLRVEREGADGSRFVFSFNRTHETVRVPVEGEVVVSSFAEVSGETISIKPNGVIVTKQ

SEQ ID NO: 11 - protein, 680 aa, Lactobacillus gasseri
MKRILNTNEFLHGGDYNPEQWWDEPDVINQDFALFKQAKINTVTVGIFSWAKLEPEEGNYDFSWLDSIFDRVEEMNGHVVLATPSGARPAWLAQKYPEVLRTDNLGNKRGFGGRHNHCLTSPIYREKVREINTKLAEHFGQRKSLVLWHISNEYSGECYCESCKNAFRDWLKNKYGNLDNLNHAWWNTFWSHTYNDWSQVNPPSPLGEMGNKGMNLDWKRFITDQTISFIDNEAAPLRKITPNVPVTTNMMAGNPLMDPFAGFDYQKVAKHLDFISWDSYPAWGNDNQTTAELGRNVGLVHDFFRSLKHQNFLVMENTPSRVNWHSVDRAKRPGMHELASLQDVARGSQGVLYFQLRASRGSSEMFHGAVIEHLHPEQTRAFKDVTTVGKDLENIRPIINTNYAKARVAIVFSYDSYWALQDAESYSKDKKIWQTIQKHYRYFYKHDIPVDFVSVEDDFSNYDLLIDPMHFLMSKAYLKKLASYVKNGGRVVGTYISGVVDENDLAYMNEWPKELQDIYGVEPLETDVLYPGQSNTLNFDGHEYKAHDYCETLINCRGKVLAKYASDFYQDTPAVVEHEYGAGKGYYLACRTDYDLLEKFYEKITANLIPEFPVKKFSSNISIQVRENKDQKYYFVQNFSDKSEQIKVDGELEDLLEKKIDRGEVVLNPFGSKIYYKKGN

SEQ ID NO: 12 - protein, 621 aa, Lactobacillus helveticus
MLEPEEGKYDFSELDKVVKKLSDANFDIVIGTSTAAMPAWMFKKYPDVARVDYQGRRHVFGQRYNFCPNSKNYQRLAGNLVEELAKHYQNNPNIVVWHVNNEYGGNCYCENCQHEFRKWLKDKYQTLDALNKAWNMNVWSHTIYDWDEIVVSNELGDAWGPEGSETIVAGLSIDYLRFQSESLQNLFKMEKQIIKKHDSEAPVTTNFHSLPNKMIDYQKWAKDQDIISYDSYHTYDAPTYKPAFLYNLMRSLKHQPFMLMESAPSQVNWQPYSPLKRPGQMAATELQAVAHGADTVQFFQLKQAVGGSEKFHSAVIAHSQRTDTRVFKELVDLGHKLKRAGSTILGSTINAKVGIVFDWSNFWSYEYVDGISQDMDYVDSILDYYRQFYERNIPTDIISVDDDFSKYDLIVAPVLYMVKDGLAEKINNYVECGGNFVTTYMSGMVDSTDNVYLGGYPGPLKNVTGIWVEESDAVVPGHTTTVSLKGKDYKAGFVCDLIHPEQAKVLAEYSNEFYAGTPAITENKYGQGKAWYVGTRLDHTGLTQLFNHIVLESNIESLVCDGDKLEVTKRVTQDGQELYFVLNMSNEVRNLPQKFIGYQDILTDKKASDKLERWGVQVLTK

SEQ ID NO: 13 - protein, 710 aa, Bifidobacterium longum
MTTHRAFRWPSLLTESGRGIAFGGDYNPDQWPEETLDEDIRLMGEAGVNVVSLAIFSWDKIEPVEGAFTFEWLDHVIDRLGRAGIAVDLASATAAAPLWLYESHPEVLPVDRYGHTVNAGSRQSWQPTSPVFKEYALRLCRKLAEHYKDNPYVTAWHMGNEYGWNNRYDYSDNALAAFRTWCEAKYGTIDALNEAWGTAFWSQHVNSFDEVLLPRHMGGDAMVNPSQQLDYERFGNDMLLDFYKAERDAIEQICPDKPFTTNFMVSTDQCVMNYAKWADEVDFVSNDHYFHEGESHLDELACSDALMDSLALGKPWYVMEHSTSAVQWKPLNTRKRAGELMRDSLAHVAMGADAICFFQWRQSKSGAEAFHSAMLPHAGADSKVFRGVCELGKALKTLSDAGLQGTELERAGTAILFSAESEWATRSETLPSMKLNHWHDVRDWYRGFLDAGLRADVVPLAYDWTGYKTIVLPTVLSLSDEDVLRIADFAKAGGTVIVGYAAGLIDEHFHIGLGGYPGAGNGLLRDMLGIRSEEFNILGEEAEGEPSEISLSNGLTTRLWQNDVTSVAADTTVLASYAGESAADWELERTPAITSRPYGNGTAIYVGCDLNRHDIAQLLKALGSRWQELSAQPTESGQTPTYPTTDPRILHTIRRSADGSTRFDFYLNRSNQPVAINGVEGDPIIAHRCETDAVGYTLNRNAILIAKTSC

SEQ ID NO: 14 - protein, 691 aa, Bifidobacterium longum
MERKEFKWPQPLAGNKPRIWYGGDYNPDQWPEEVWDEDVALMQQAGVNLVSVAIFSWAKLEPEEGVYDFDWLDRVIDKLGKAGIAVDLASGTASPPMWMTQAHPEILWVDYRGDVCQPGARQHWRATSPVFLDYALSLCRKMAEHYKDNPYVVSWHVSNEYGCHNRFDYSEDAERAFQKWCEKKYGTIDAVNDAWGTAFWAQRMNNFSEIIPPRFIGDGNFMNPGKLLDWKRFSSDALLDFYKAERDALLEIAPKPQTTNFMVSAGGTGIDYDKWGYDVDFVSNDHYFTPGEAHFDELAYSASLCDGIARKNPWFLMEHSSSAVNWRPINYRVEPGELVRDSLAHLAMGSDAICYFQWRQSKAGAEKWHSSMVPHAGPDSQIFRDVCELGADLNKLADEGLLSTKLVKSKVAVVFDYESQWVTEHTATPTQEVRHWTEPLAWFRALADNGLTADVVPVRGSWDEYEAVVLPSLTILSEETTRRVREYVANGGKLFVTYYTGLVDDKDHVWLGGYPGSIRDVVGVRVEEFAPMGNDFPGAMDHLDLDNGTVAHDFADVITSVADTAHVVASFKADKWTGFDGAPAITVNDFGDGKAAYVGARLGREGLAKSLPALLEELGIETSAEDDRGEVLRVERADETGENHFVFLFNRTHDVAVVDVEGEPLVASLAQVNESERTAAIQPNGVLVVKL

SEQ ID NO: 15 - protein, 719 aa, Bifidobacterium longum
MTTRRTFRWPSLLTESGRGIAFGGDYNPDQWPEETLDEDIRLMVQAGVNTVALAIFSWDKIEPREGEFTFEWLDHVIDKLGAASIAVDLASATATAPLWLYERHPEVLPIDRYGHVVNAGSRQSWQPTSPVLKEYALRLCRKLAEHYKDNPYVTAWHMGNEYGWNNRYDYSDNALAAFRTWCEAKYGTVDALNEAWGTAFWSQHVNSFDEVLLPRHMGGDSMVNPPQQLDYERFGNDMLLDFYKAERDAIEEICPGKPFTTNFMVSTDQCTMDYAQWANEVDFVSNDHYFHEGESHLDELACSDALMDSLALGKPWYVMEHSTSAVQWKPLNTRKRAGELMRDSLAHVAMGADAINFFQWRQSASGAEAFHSAMVPHAGSDTKLFRGVCELGAALKTLSDAGVQDTELKRADTAILFSAESEWATRSETLPSMKLNHWHDVRDWYRGYLDAGARADVVPLAYDWSGYQTIVLPTVIALSDEDTRRIADFAENGGTVIVGYATGLIDEHFHIGLGGYPGAGNGLLRDMLGIRSEEFNILGEEAEDEPAEIGLSNGLTTRLWQNDVTSVAPDTRVLATYVGTAAADWELDGVPAITSHPHGQGAAIYVGCDLGRHDITHLLKELNTTAPSDERAPDQRPGGGEINAATTTAAATTHDPRILHTIRQSSDGTIRFDFYLNRSKQPVAVNGVEGDPIIAHRCETDAVGYTLNRNAILIAKTSC

SEQ ID NO: 16 - protein, 672 aa, Lactobacillus reuteri
MMKKELPRFLYGGDYNPEQWPEETWDEDIKVFKQADINSATINVFSWALLEPQEGKYDFTKLDKIIKELTVADFDIVLATSTAAMPAWMFKKYPDVARVDYQGRRHVFGARHNFCPSSKNYRRLAKNLVEQLAKRYGDNPHIVAWHVNNEYGGNCYCEECQTEFQQWLKARYQTLDNLNHAWNMNVWSHTIHDWNEIVVPNELGDAWGPEGSETIVAGLSIDYLRFQSAQMLDLFKMEKQIIEKYDPTTLVTTNFHSLPNKMIDYQQWASAQDIISYDSYPAYDAPIYQPAFLYDLMRSLKHQPFMLMESTPSQVNWQPYSPLKRPGQMAATELQAVAHGADTVQFFQLKQALGGSEKFHGAVISHANRTDTRVFKEVAKLGHDLRKVGPVIKDSQTKARVALIFDWSNFWSFEYVDGITQDLKYVPIILDYYRQFYELNIPTDVISVDDDFRQYDLVVAPVLYMVKGGLGKKITDYVANGGNFITSFMSGMVNESDNIYPGGYPGPLKDVMGLWVEESDAILPNKDVKLTMTTGDELTGYLIADLIRLNGAHVLAEYASEFYAGTPAVTENTYSKGKAWYVGSRLDHASLRKIIMHIVDDVHLSALVKEPTELEITKRQNSAGQDIYFVLNMGKGKQPLPVEFQKGYRDLLTGDSPETMLDSWDVEILVQE

SEQ ID NO: 17 - protein, 1007 aa, Lactobacillus delbrueckii
  source 1..1007: note = subsp. lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLGFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLASLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 18 - protein, 705 aa, Bifidobacterium angulatum
MAHRRTFHWPSLLTESGRGIAFGGDYNPDQWPEDVWDDDIRLMKQAGVNTVALAIFSWDRIQPEKHRWEFGWLDCIIDKLGKAGIAVDLASATATAPLWLYEQHPEVLPHDKYGHPINAGSRQSWSPTSPVFKEYALTLCRKLAERYGTNPYVTAWHMGNEYGWNNRYDYCDNALHAFRAWCERKYGTIEALNAAWGTTFWGQEMNGFDEVLIPRFMGADSMVNPGQKLDFERFGNDMLLDFYRAERDAIAEICPDKPFTTNFMVSTDQCCMDYADWANEVDFVSNDHYFHEGESHIDELFCSDALMDSLALGRPWYVMEHSTSAVQWKDLNIRKRKGETVRDSVAHVAMGADAINFFQWRASAFGAESFHSAMVPHAGEHTKLYRSVCELGAALKTLGDAGVQGSELVRSDTAILFSAESEWATRSETLPSKKLNHWHDVRDWYRAYLDAGTRADIVPLKYDWSGYATVVLPTVLMLSAADTARLERFVRDGGTVVVGYASGLIDENFHTWLGGYPGAGDGMLRTMLGIRGEEFNILGAQAEGEPSEIRLSNGMVTRLWQNDIAVDGADTEVLASYAGTQADEWELDGTAAITRNPYGKGMAYFVGCDLNVADLAVFVGDHLTVGQACEAGDGADYDPTITLHTERASAEAIFDFYLPRGKNETVVSGISGEPVYRFQCDEGEAPGVYTIRRNGVLVVKRYNRQ

SEQ ID NO: 19 - protein, 626 aa, Lactobacillus fermentum
MEAELKWLDDPEVFRVNQLPAHSDHRFYRDQEEAALEKSSYVQNLNGRWGFKFSKNPMERPVDFYKLDFDRNDFGEIEVPSEIELSNFAQINYTNITMPWTGKIYRRPAYTLGDNKEEGSFSQGQDNTVGSYVRHFTLAEGLKNHDVHVVFEGVERAMYVWLNGHFIGYAEDSFTPSEFDLTPYLVDGDNLLAVEVYKHATSSWIEDQDMFRFSGIFRDVNLVAQPSIHVQDLKINARVADDMKTGSLGLVLKMVGQPGSVQVEVADQTGAAVLNRQLNADGNWTMAPVQLVGIHLWDNHHPYLYQLTLTVRDATGRVVEVIPYQFGFRRVEIDQDKVLRLNGKRLIINGVNRHEWNCHRGRAVTIEDMHTDLGIFKENNINAVRTSHYPDQIPWYYLCDREGIYMMAENNLESHATWQKFGQDEPSYNVPGSLPQWKEAVVDRARSNYEIFKNHTAILFWSVGNESYAGEDILAMNNYYKEVDDTRPVHYEGVVHTKEYRDQISDFESWMYLPPKEVEAYLKKNPDKPFIECEYMHSMGNSVGGMGSYIKLLDKYPQYCGGFIWDFVDQAIEVVDPVTGQKSMRYGGDFDDHHADNEFSGDGICFADRTPKPAMQEVKYYYGLHK

SEQ ID NO: 20 - protein, 320 aa, Lactobacillus fermentum
MDYTNKLHVVYDDNILGLDGKDFQYLFSYEQGGPESFKIKGKEWLYRSPRPTFWRATTDNDRGNGFNVSSVQWLAADYVLPCQDIALQVDGKDKKLPLAPKTNRYSNQEFAKKVKITFTYQTQTVPATTVQVSYTVKASGKIKVNVHYTGAQLPSLPVLGWRMIMPTPATSFDYEGLSGETYPDRMAGGIEGTYHVEGLPVTPYLVPQENGMHMANKWVQITRATTLNNADPDAAPFRLKFEAPKKGKLNFSCLPYTSAELENATHPEELPAAHRTVLVIAGEVRGVGGIDSWGADVEEKYHIDATVDHDFSFKIVPELN

SEQ ID NO: 21 - protein, 1305 aa, Bifidobacterium bifidum
MVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTEAAANITLKQTVFPKGGKTDAAIGTVTTASKSIAAGASADVTSTITAASPKLWSIKNPNLYTVRTEVLNGDTVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQTIAGDNAVLGGDKDETWAKFDLTSTINRDRNAPSVIMWSLGNEMMEGISGSVSDFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNKVPVVVYTDAAKVKLYFTPKGSTEKRLIGEKSFTKKTTAAGYTYQVYEGTDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD